SEQUENCE LISTING (9 sequences)

SEQ ID NO: 1 - DNA, 5390 bp, Homo sapiens
ctctcatcaatgagagtgatatttgggggttgaaatcagtgttctggctcttaaatatgattagatggacttattgtgtgggaggtgaataaagccaagataaaagacaataatttcattcagaagaaaatatagctgaatagatgtaatttcagtcaattaatacaggatgggttggtacacatataacactatatccacttgtttaaaaaattgcttaggtgttgaaaaattgctgtggcacttcctggaaagtaatttttaatacaggtaaatgggtgagtcttgtgtaaagtcagaaagagcaccttggaaaatgcctgaattttcctttcaaatatatttataaccaatcttcaaataatatttttctcagactggaaatttgtattctaaattatttttagaaaaaaaaaatccccaccccctccagaattaaaacacctgtaaaattcaggattatattggaagtgttgacatggttttaagggtagcaattgcaaaagtgctactaaaatatattattacaggtatatttttatatcagttaattggtacaccaacccttctgaaagagtgttaaagaagaagcataaagtataacaccttgagtccatgatatattttggggaagaaacctcttgcaatctttaaattagcatgaaaagctacaccaccaagatttccatagcaaagccattggatgaagcctcccactcaatgtgacataaaagtaattcctgaaactttggaaagtcaataaaaatatatggaacattaagtagaggtaaagaagggattgtaactaaacttagaataacgaattaaaactctcccatggctttgatatggcaactataaaatgtagggttaccacaaagtttgaaattatagaatactatagtattcttaagaaatattatgacattatatttttaaggtttaattaggtaataagatttgatcctttgatggtatattcagttattcaacggacaatatacgtctacttgtacttttataagtaaatatttatatataccggaggtcaatttctgtttctgtggtaactgccctacaggtaagcttagcagcttttaatgaagtagaattgaaatacttttgggggaaagtcacacttctatttaagtatatgtatcttttttcgggattatgtcatagtgcaatgtaaatcttagttatggtatttagaatgtcagaatgtcacattttgtgctgatttgcatacttcaatagtctggagcagttgaagcagctaaactatttagctctgggaaggttggaattcccgtaagaattatagtgatctatgtgtaacataacatgagaatcagacttcctgaagtatcaactactagaggtattactaataacaacgggccaaaaaagaggaaaaacttcttcaaaggataatggatcagtacttttagccaatttgtgattttaaattttgcctgatggagttcaatttcactctagtcgaggcattcttgttcttcaccactggcctttctgccatccattctcttttctttatggctgttgcaaattttcctcatctcttcttcatacttgataaaattctccccaaaccttgtccaagctttgaatggaacagtaatagtattacggtaaggtggtctcacctcacttaccttcaggaaaattccatatttattagagcccacatcaaagtagaaccttttattgtccactctgaaagaagtcccctctgggagttcaagcgggtcatcgtctccacctcttcgttcttctatgtctccttcgccatagtcttcaatcagctgaaccaaggcatcacgaaactcaatcattccttgtgctgggaggacaatagtctgttcttggcccaaactgtggccaaaataacctatcatgccagtcccccgcatcatggtttgtctaatccgtaggaagcgaccccgctgattttcctttaggtctaggtaatatttcctattgtccctctcgatatagtctgttttcaggacactgtgaggatgctcttcggaccccaccgagactggtggggagggtgccgagtgcttctgcctccttctggagccttgctctttgctgtggccatgctcttgccggtggcctttcaggcccaggtgggcatagtgctcgatgaagtcccctagacagtccttcagctccgctgccacagacagggagagggtcagtttactctttctgatgttgtcctgccggcctctccctatccagacttcggctatctttaggaagcggccccgggagctttgcttcacgtctaggtaaaacctctttttctggatgtccactcgtttggaggccagctcctggatttcggctgcgcccccggcctgattaggggtggctgaggccgcgtagtgggggtagtgggagtgctgggcctggggatagagtctactcttgcttaggccagagccccctacattcttgcctccgcggccgcggccgccgccgcctccccttcgcctggctctttccatcttcagctgcaagtaacaaacagacacacgggatggggtgggggaggggtgttgagaacaatcgcagacgcccctcggcctgaccgccccgccgccgcccgcgacccccacgccgggcccggctcccccggcgccgcagcgcggggctccagccactgccggccggttggcggccgccgctccggctctgcccgcgctccccgcatccctccgccgcccccggggccgctcccgcacgccgccgccgcccgtcccgcgctccgcggccccaggtctccagccggcgggcactcacatcatctctgccatcaccgccgccgccgatgcccttcacgaccaccgccgccgccaccgccagctctcggcccctctgctgcagccgccgcagccgccgccccccgcctcctcccccgccgccgccgctcgcactgccccccgccggagcagccgggcaggggcatcgcccgcggcgcccaccgcagccgcccctcctgcggccgctgcgggggccgccgcctgacttcggacaccggccccgcacccgccaggaggggagggaaggggaggcggggagagcgacggcggggggcgggcggtggaccccgcctcccccggcacagcctgctgaggggaagagggggtctccgctcttcctcagtgcactctctgactgaagcccggcgcgtggggtgcagcgggagtgcgaggggactggacaggtgggaagatgggaatgaggaccgggcggcgggaatgttctcacttctccggattccaccgggatgcaggactctagctgcccagccgcacctgcgaagagactacacttcccgaggtgctcagcggcagcgagggcctccacgcatgcgcaccgcggcgcgctgggcggggctggatgggctgtggtgggagggttgcagcgccgcgagaaaggcgagccgggccgggggcggggaaaggggtggggcaggaacgggggcggggacggcgctggaggggcgggtcgggtaggtctcccggagctgatgtgtactgtgtgcgccggggaggcgccggcttgtactcggcagcgcgggaataaagtttgctgatttggtgtctagcctggatgcctgggttgcaggccctgcttgtggtggcgctccacagtcatccggctgaagaagacctgttggactggatcttctcggggtaaagtgtcttcctattcccgttttcccagctccgtgccccggggtatctatagtgctaggcttctgagacacgtggaggcgcttaggcccgcggagcaggcccctgggctgtgtttgggagattctggaaaaacagactttctcccttttcctttcccctctggagcagctgggaaaggcgtctctcggagcttccgccattcagaaagtgtttccccgcccctgacggccccagcttgatgtttaccttgcgcgcttagttaggattgctctttctagtacggcctttattatggcagaaattgagccactacaggtctgcacagcaaagtcgagggaaataacctgctgtctatgatttagcaagtgatgatggatatttttgagttcaaatcactcagtctttaatggattttcttttctttttttcctagttgaatctcagttgttctcaggctaggtttgatatgaaaaatactactccctattgaagagggtaatataattcttaggatgctaatacaggagtctgtaagtcggagtacttcaggctttttatggttcggtacatttctttttaggggtgggatattgggataagtaggacagttcagatcttgatgtcattgaatagtggtgtctacctaaaaggaagaggctggggcacaaaattataatttaaagtgtttacttcagccaaaatgaggacagctgcccaggactcatttcccagttgccttggggagtgctccttcagccttggttacaagcaggtttttaaaggcgaaggggaagagtgggctgagacaaagttggtcaggaattgttattggtttacagagataccattgcttagtgattggctgtacactgtaactcatagggtatgagttatcgtgtccagcatatggcattttatggcttcttggttccagtctagagcccatatagcaggtgggctttaagagataattatccagctcagaccgggagtgagacgggactgttttttcattctaatgcctctctgggcctgataatttaaaggggcttgcattcttcagctaaaattttattttcttttattttttttcccccacagtggtctccctttattaatccaacaaatgtttattaaggactgctctaggcactagagataccaacaatgaacaaaacagacaaacatctctctccttttggagcttacattttagtggaaagagacaggcaaaaagtaaaattataggatgacagaaggtgaaaatagagattaaaaaggaaagggggatgaagagtgtgtgtgtgtgttgtgggtggggggctgtagggagctgcagttttacaataagtggtcagggacgtcctcgctgagaagggaatttttgagtccagagctcaaggaagtgagagaacttaccttgacgttctctggaggagaagaaagtacaaaggccctgggggaacctgcctctttatgttcagagagcaacttaagaggcctagtgagctggagcagagcggagaggaggtcagaggtcaggccagaaagggaaaggggccagaatgtgtaggactttatgggctatattgaaaattggggctttcattctgagggagatgggaagctgttggagagttgttagggaaacgtgat

SEQ ID NO: 2 - DNA, 25 bp, Artificial Sequence
  Description of Artificial Sequence Synthetic primer
gtagttgggtaggggtattgtttgt

SEQ ID NO: 3 - DNA, 23 bp, Artificial Sequence
  Description of Artificial Sequence Synthetic primer
aaacaaaatccaccacccacccc

SEQ ID NO: 4 - DNA, 20 bp, Artificial Sequence
  Description of Artificial Sequence Synthetic primer
cgggtaggggtatcgttcgc

SEQ ID NO: 5 - DNA, 20 bp, Artificial Sequence
  Description of Artificial Sequence Synthetic primer
aacgaaatccaccgcccgcc

SEQ ID NO: 6 - DNA, 24 bp, Artificial Sequence
  Description of Artificial Sequence Synthetic primer
aggtttttagtyggygggtattta

SEQ ID NO: 7 - DNA, 18 bp, Artificial Sequence
  Description of Artificial Sequence Synthetic primer
aaccccctcttcccctca

SEQ ID NO: 8 - DNA, 22 bp, Artificial Sequence
  Description of Artificial Sequence Synthetic primer
gcatgtgttcggaagagtgttt

SEQ ID NO: 9 - DNA, 21 bp, Artificial Sequence
  Description of Artificial Sequence Synthetic primer
tgacatggaagaaacgtggaa

(19) United States
(12) Patent Application Publication
(10) Pub. No.: US 2009/0047214 A1
(43) Pub. Date: Feb. 19, 2009

(54) Novel methylation marker

(75) Inventor: Manel Esteller, Madrid (ES)

Correspondence Address: FOLEY AND LARDNER LLP;SUITE 500, 3000 K STREET NW, WASHINGTON, DC, 20007, US

(73) Assignee: OncoMethylome Sciences S.A.

(21) Appl. No.: 11/798,312

(22) Filed: May 11, 2007

Related U.S. Application Data
(60) Provisional application No. 60/747,139, filed on May 12, 2006.

(30) Foreign Application Priority Data
May 12, 2006 (GB) 0609498.1

(51) Int. Cl. A61K 51/04; C12Q 1/68; C12Q 1/02; A61K 31/711; C07H 21/00; A61K 33/24; A61K 31/4375

(52) U.S. Cl. 424/1.73; 435/6; 435/29; 514/283; 424/649; 514/44; 536/24.33

(57) ABSTRACT

A method for predicting the likelihood of successful treatment of cancer with a topoisomerase inhibitor and/or a DNA damaging agent and/or a DNA methyltransferase inhibitor and/or a HDAC inhibitor comprises the steps of:

(a) determining the methylation status of a RecQ helicase family gene in a sample obtained from a subject, wherein if the RecQ helicase family gene is methylated the likelihood of successful treatment is higher than if the RecQ helicase family gene is unmethylated; and/or
(b) determining the expression levels of a RecQ helicase family gene in a sample obtained from a subject, wherein a reduced level of expression of the RecQ helicase family gene indicates the likelihood of successful treatment is higher than if the RecQ helicase family gene is expressed at a higher level. Corresponding kits are also useful in predicting the likelihood of successful treatment of cancer. The most preferred RecQ helicase family member is WRN. Related methods and kits are also useful for diagnosing cancer, predicting resistance to certain treatments and for determining treatment regimens. Methods of treatment of cancer may involve using the aforementioned methods and also reconstituting RecQ helicase family member activity in a subject.

### CROSS-REFERENCE TO RELATED APPLICATIONS

This application is the non-provisional of U.S. Provisional Application No. 60/747,139, filed May 12, 2006, which claims priority to Great Britain Patent Application No. 0609498.1, filed May 12, 2006. The contents of these applications are incorporated herein by reference in their entirety.

### FIELD OF THE INVENTION

The present invention relates to methods and kits for diagnosing cancer which include determining the methylation status or expression levels of a RecQ-family helicase gene, in particular the WRN gene. Also described are pharmacogenetic methods for determining suitable treatment regimens for cancer and methods for treating cancer patients, based around selection of the patients according to the methods of the invention.

### INTRODUCTION

Werner Syndrome (WS) is an inherited premature aging disorder which is characterized by genomic instability and by a high incidence of malignant neoplasms (Epstein et al., 1966; Salk, 1982). Mutations in the WS gene (WRN) are found in patients exhibiting the clinical symptoms of WS (Yu et al., 1996; Goto et al., 1997; Yu et al., 1997).

The vast majority of WRN mutations result in loss of function of the WRN protein (Matsumoto et al., 1997). The WRN protein has been demonstrated to possess helicase and exonuclease activities (Gray et al., 1997; Suzuki et al., 1997; Huang et al., 1998) and cultures of WS cells show increased chromosomal instability with abundant deletions, reciprocal translocations and inversions (Thweatt and Goldstein, 1993; Opresko et al., 2003). WRN belongs to the RecQ family of helicases, highly conserved from bacteria to man, which members are proposed to be essential caretakers of the genome (Opresko et al., 2003; Hickson, 2003). Evidence suggests that the Werner syndrome protein (WRN) contributes to the maintenance of genome integrity through its involvement in DNA repair. In particular, biochemical evidence indicates a role for WRN in base excision repair (BER).

In addition to WRN, germline mutations of two other RecQ helicases, BLM in Bloom syndrome and RECQL4 in Rothmund-Thomson syndrome are also associated with an elevated incidence of cancer (Hickson, 2003). Since patients with WRN germline mutations develop with high penetrance a broad spectrum of epithelial and mesenchymal tumors, being one of the main causes of death of these patients before the age of 50, a tumor-suppressor function for WRN has been proposed. This putative role is further supported by a very high rate of loss of heterozygosity (LOH) at the chromosomal WRN loci at 8p12-p11.2 across many tumor types, including colorectal and breast cancer (Chughtai et al., 1999; Armes et al., 2003). However, somatic mutations of WRN have not been described in sporadic neoplasms.

### SUMMARY OF THE INVENTION AND DEFINITION OF TERMS

The present invention is based around the finding that the RecQ-family helicase, WRN undergoes CpG island promoter methylation-associated gene silencing in human cancer cells. The hypermethylation of the WRN promoter leads to its loss of expression and hypersensitivity to topoisomerase inhibitors and DNA damaging agents. The epigenetic loss of WRN function can be rescued by the use of DNA demethylating agents. Furthermore, the reintroduction of WRN into those transformed cell lines with WRN-deficiency due to hypermethylation provokes a reduction in colony formation and a decrease in growth of tumor xenografts, supporting the hypothesis of a tumor-suppressor role for WRN. The analysis of a large panel of human primary tumors shows that WRN CpG island hypermethylation is a common event in tumorigenesis.

Importantly, for cancers such as colorectal cancer, the presence of aberrant methylation at the WRN promoter predicts improved survival in those patients treated with topoisomerase inhibitors such as irinotecan, a treatment commonly used in this neoplasm. These findings, as discussed in the experimental section below, underline the significance of WRN as a caretaker of our genome with tumor suppressor activity, and identify epigenetic silencing of WRN as one key step in cancer development that may have an important clinical impact for the treatment of these patients.

Accordingly, in a first aspect, the invention provides a method for predicting the likelihood of successful treatment of cancer with a topoisomerase inhibitor and/or a DNA damaging agent and/or a DNA methyltransferase inhibitor and/or a HDAC inhibitor comprising determining the methylation status of a RecQ helicase family gene in a sample obtained from a subject, wherein if the RecQ helicase family gene is hypermethylated the likelihood of successful treatment is higher than if the RecQ helicase family gene is unmethylated, or methylated to a lesser degree. Preferably, the methylation status of at least the WRN gene is determined.

The opposite scenario is also envisaged in the present invention. Thus, in a related aspect, the invention provides a method for predicting the likelihood of resistance to treatment of cancer with a topoisomerase inhibitor and/or a DNA damaging agent and/or a DNA methyltransferase inhibitor and/or a HDAC inhibitor comprising determining the methylation status of a RecQ helicase family gene in a sample obtained from a subject, wherein if the RecQ helicase family gene is unmethylated the likelihood of resistance to treatment is higher than if the RecQ helicase family gene is methylated. Thus, the detection of an unmethylated RecQ helicase family gene indicates the probability of successful treatment with a topoisomerase inhibitor and/or a DNA damaging agent and/or a DNA methyltransferase inhibitor and/or a HDAC inhibitor is low. Again, preferably, the methylation status of at least the WRN gene is determined.

Since methylation of a RecQ helicase family gene, in particular the WRN gene, manifests itself in down regulation of gene expression, the invention also provides a method for predicting the likelihood of successful treatment of cancer with a topoisomerase inhibitor and/or a DNA damaging agent and/or a DNA methyltransferase inhibitor and/or a HDAC inhibitor comprising determining the expression levels of a RecQ helicase family gene in a sample obtained from a subject, wherein a reduced level of expression of the RecQ helicase family gene (as caused by methylation of the gene) indicates the likelihood of successful treatment is higher than if the RecQ helicase family gene is expressed at a higher level (because the gene is unmethylated). Preferably, the expression levels of at least the WRN gene are determined.

The opposite scenario is also envisaged in the present invention. Thus, in a related aspect, the invention provides a method for predicting the likelihood of resistance to treatment of cancer with a topoisomerase inhibitor and/or a DNA damaging agent and/or a DNA methyltransferase inhibitor and/or a HDAC inhibitor comprising determining the expression levels of a RecQ helicase family gene in a sample obtained from a subject, wherein normal levels of expression of the RecQ helicase family gene (as caused by a lack of methylation of the gene) indicates the likelihood of resistance to treatment is higher than if the RecQ helicase family gene is expressed at a lower level (because the gene is methylated). Thus, the detection of a lack of down regulated expression of the gene taken from the RecQ helicase family, due to a lack of methylation of the gene, indicates the probability of successful treatment with a topoisomerase inhibitor and/or a DNA damaging agent and/or a DNA methyltransferase inhibitor and/or a HDAC inhibitor is low. Again, preferably, the expression levels of at least the WRN gene are determined.

The RecQ family of helicases have been characterised in the art (see, for example, Opresko et al., 2003 and references cited therein). Thus, by “RecQ helicase family gene” is meant any gene which is taken from the RecQ helicase family. Non limiting examples of RecQ helicase family members which may be assessed, either in terms of their methylation status or expression levels as determined by their methylation status, according to all aspects of the invention include RecQL, BLM (BLOOM), WRN, RecQ4 and RecQ5.

RecQL has two transcript variants, the first having the accession number NM_{—}032941 and the second NM_{—}002907. The BLM gene sequence has the accession number NM_{—}000057, for RecQL4 the accession number is NM_{—}004260 and for RecQL5 it is NM_{—}001003716 Most preferably, the methylation status and/or expression levels of the WRN gene is/are determined according to the methods of the invention. The WRN gene sequence has the accession number NM 000553.

By “likelihood of successful treatment” is meant the probability that treatment of the cancer using any one or more of the listed therapeutic agents will be successful.

“Successful treatment” is defined to include complete recovery and also significant tumour regression, prevention of metastasis and improved survival rates. Improved alleviation of symptoms may also be considered as “successful treatment” in the present invention.

“Resistance” is defined as a reduced probability that treatment of cancer will be successful using any one of the specified therapeutic agents and/or that higher doses will be required to achieve a therapeutic effect.

“Cancer” is defined to include neoplasias, in particular malignant neoplasias.

By “topoisomerase inhibitor” is meant any molecule, compound or otherwise which is capable of inhibiting topoisomerase activity and thus acting as an anti-cancer agent. Topoisomerases are enzymes that control the topology of supercoiled DNA helices during replication. There are two major types, topoisomerase I which initiates cleavage of a single DNA strand and topoisomerase II which cleaves both strands. Although not bound by this theory, inhibiting topoisomerase activity is thought to prevent replication of cancer cells causing cell death, thus leading to the effective treatment of cancer.

“DNA damaging agent” is defined to include all agents which are capable of causing sufficient damage to DNA in cancer cells to cause cell death, or at least prevent or disrupt replication and/or growth of cancer cells. Cancer cells in which the WRN gene is hypermethylated have been shown to be particularly susceptible to DNA damage (see, for example, the chromosome breakage results discussed below).

By “DNA methyltransferase inhibitor” is meant any molecule, compound or otherwise which is capable of inhibiting DNA methyltransferase activity and thus acting as an anti-cancer agent. DNA methyltransferases catalyze transfer of a methyl group to DNA. In particular, DNA methyltransferases are responsible for methylation of DNA at CPG sites, which can cause down regulation of important tumour suppressor genes, such as WRN. Although not bound by this theory, inhibiting DNA methyltransferase activity is thought to prevent down regulation of important tumour suppressor genes through methylation, thus leading to the effective treatment of cancer. Inhibitors of DNA methyltransferase activity may influence expression of the DNA methyltransferases or may inhibit the enzymatic activity of the protein for example.

“HDAC inhibitor” is defined as any molecule, compound or otherwise capable of inhibiting histone deacetylase activity and thus acting as an anti-cancer agent. Histone deacetylases are a class of enzymes which are responsible for removal of acetyl groups from lysine amino acids in histones. This removal of an acetyl group means that the positively charged lysine becomes available for interaction with DNA. The interaction of histones with DNA generally down regulates gene expression by blocking access to the DNA of components required for transcription. Many HDAC inhibitors are in clinical trials and are known to be useful in treating cancer. Inhibitors of HDAC activity may influence expression of the HDACs or may inhibit the enzymatic activity of the protein for example.

By “methylation status” is meant the level of methylation of cytosine residues (found in CpG pairs) in the WRN gene which are relevant to the regulation of gene expression. Thus, the levels of methylation of the WRN gene are determined by any suitable means in order to reflect whether the gene is likely to be down regulated or not. Generally, an increase in methylation is associated with a corresponding decrease in gene expression.

“Expression levels” are defined to include levels of both mRNA and protein produced by transcription of the appropriate gene and translation of the mRNA produced by transcription of the gene respectively. Changes in the level of expression may be measured directly or indirectly. Indirect measurement may involve determining expression of genes whose expression is modified or at least partially determined by activity of the relevant RecQ family helicase.

“Hypermethylation” is a well-known term in the art. It is defined as an increase in the level of methylation above normal levels. Thus, it relates to aberrant methylation at specific CpG sites in a gene, often in the promoter region. Normal levels of methylation may be defined by comparison to non-cancerous cells for example. Methylation and hypermethylation are generally linked to down regulation of gene expression. In this invention, methylation and in particular hypermethylation of the WRN gene is indicative of a loss of expression of this tumour suppressor gene which provides a reliable indicator of cancer.

In a related aspect, the invention provides a method of selecting a suitable treatment regimen for cancer comprising determining the methylation status of a RecQ helicase family gene, preferably the WRN gene, in a sample obtained from a subject, wherein if the RecQ helicase family gene, preferably the WRN gene, is hypermethylated a topoisomerase inhibitor and/or a DNA damaging agent and/or a DNA methyltransferase inhibitor and/or a HDAC inhibitor is selected for treatment.

The opposite scenario is also envisaged in the present invention. Thus, in a related aspect, the detection of an unmethylated RecQ helicase family gene, preferably the WRN gene indicates that treatment with a topoisomerase inhibitor and/or a DNA damaging agent and/or a DNA methyltransferase inhibitor and/or a HDAC inhibitor is contra-indicated. Accordingly, alternative treatments should be explored.

As aforementioned, changes in methylation status are causally linked to changes in gene expression. Accordingly, the invention also provides a method of selecting a suitable treatment regimen for cancer comprising determining the expression levels of a RecQ helicase family gene in a sample obtained from a subject, wherein a reduced level of expression of the RecQ helicase family gene (as caused by increased methylation of the gene) indicates a topoisomerase inhibitor and/or a DNA damaging agent and/or a DNA methyltransferase inhibitor and/or a HDAC inhibitor should be selected for treatment. Again, preferably, the expression levels of at least the WRN gene are determined.

The opposite scenario is also envisaged in the present invention. Thus, in a related aspect, the detection of normal levels of expression of a RecQ helicase family gene (due to a lack of methylation of the gene) indicates that treatment with a topoisomerase inhibitor and/or a DNA damaging agent and/or a DNA methyltransferase inhibitor and/or a HDAC inhibitor is contra-indicated. Accordingly, alternative treatments should be explored. Again, preferably, the expression levels of at least the WRN gene are determined.

“Suitable treatment regimen” is defined to include the choice of treatment which is to be made by the individual carrying out the method. The regimen chosen is one deemed suitable on the basis of the methylation status and/or expression levels of the relevant RecQ helicase family gene and is selected from a topoisomerase inhibitor and/or a DNA damaging agent and/or a DNA methyltransferase inhibitor and/or a HDAC inhibitor according to the invention. Of course, as would be readily appreciated by the skilled practitioner, the specific dosage regime may be calculated according to the body surface area of the patient or the volume of body space to be occupied, dependent on the particular route of administration to be used. The amount of the composition actually administered will, however, be determined by a medical practitioner based on the circumstances pertaining to the disorder to be treated, such as the severity of the symptoms, the age, weight and response of the individual.

In an additional aspect of the invention there is provided a method of diagnosing cancer comprising determining the methylation status of a RecQ helicase family gene, in particular the WRN gene, in a sample obtained from a subject, wherein hypermethylation of the RecQ helicase family gene, in particular the WRN gene, is indicative of cancer.

Also provided is a method of diagnosing cancer comprising determining the expression levels of a RecQ helicase family gene, in particular the WRN gene, in a sample obtained from a subject, wherein a reduced level of expression of the RecQ helicase family gene, in particular the WRN gene is indicative of cancer.

“Diagnosis” is defined herein to include monitoring the state and progression of the disease, checking for recurrence of disease following treatment and monitoring the success of a particular treatment. The tests may also have prognostic value, and this is included within the definition of the term “diagnosis”. The prognostic value of the tests may be used as a marker of potential susceptibility to cancer. Thus patients at risk may be identified before the disease has a chance to manifest itself in terms of symptoms identifiable in the patient.

In a further aspect, the invention provides a method of treating cancer in a subject comprising administration of a topoisomerase inhibitor and/or a DNA damaging agent and/or a DNA methyltransferase inhibitor and/or a HDAC inhibitor, wherein the subject has been selected for treatment on the basis of measuring the methylation status of a RecQ helicase family gene, in particular the WRN gene according to any of the methods of the invention.

Similarly, the invention provides a method of treating cancer in a subject comprising administration of a topoisomerase inhibitor and/or a DNA damaging agent and/or a DNA methyltransferase inhibitor and/or a HDAC inhibitor, wherein the subject has been selected for treatment on the basis of measuring the expression levels of a RecQ helicase family gene, in particular the WRN gene according to any of the methods of the invention.

In a related aspect, the invention also provides for the use of a topoisomerase inhibitor and/or a DNA damaging agent and/or a DNA methyltransferase inhibitor and/or a HDAC inhibitor in the manufacture of a medicament for treating cancer in a subject, wherein the subject has been selected for treatment on the basis of measuring the methylation status of a RecQ helicase family gene, in particular the WRN gene according to the methods of the invention.

Similarly, the invention also provides for the use of a topoisomerase inhibitor and/or a DNA damaging agent and/or a DNA methyltransferase inhibitor and/or a HDAC inhibitor in the manufacture of a medicament for treating cancer in a subject, wherein the subject has been selected for treatment on the basis of measuring the expression levels of a RecQ helicase family gene, in particular the WRN gene according to the methods of the invention.

In a still further aspect the invention provides a method for treating cancer in a subject, said subject having a reduced level or activity of a helicase from the RecQ family, in particular WRN, comprising reconstitution of helicase, in particular WRN, activity in the subject. Preferably, the reduced level or activity of the helicase from the RecQ family, in particular WRN is detected by determining the methylation status of the RecQ helicase family gene, in particular the WRN gene in accordance with the methods of the invention, although it may be determined directly in accordance with the methods of the invention.

In a related aspect, the invention provides for the use of a vector carrying a RecQ helicase family gene, in particular the WRN gene in the manufacture of a medicament for treating cancer in a subject, wherein the subject has been selected for treatment by determining the methylation status of the RecQ helicase family gene, in particular the WRN gene in accordance with the methods of the invention.

Similarly, the invention provides for the use of a vector carrying a RecQ helicase family gene, in particular the WRN gene, in the manufacture of a medicament for treating cancer in a subject, wherein the subject has been selected for treatment by determining the expression levels of the RecQ helicase family gene, in particular the WRN gene in accordance with the methods of the invention.

By “treatment” is meant a decrease in the severity of symptoms associated with the disease and/or inhibition, which may be partial or complete, of progression of the disease. An increase in survival, to include increased survival time and/or increased rates of survival, is also considered to be effective “treatment”. Prevention of metastasis of a cancer may also be considered as an effective “treatment”.

### DETAILED DESCRIPTION OF THE INVENTION

As aforementioned, the invention provides in a first aspect a method for predicting the likelihood of successful treatment of cancer with a topoisomerase inhibitor and/or a DNA damaging agent and/or a DNA methyltransferase inhibitor and/or a HDAC inhibitor comprising:

(a) determining the methylation status of a RecQ helicase family gene in a sample obtained from a subject, wherein if the RecQ helicase family gene is hypermethylated the likelihood of successful treatment is higher than if the RecQ helicase family gene is unmethylated; and/or
(b) determining the expression levels of a RecQ helicase family gene in a sample obtained from a subject, wherein a reduced level of expression of the RecQ helicase family gene (as caused by methylation of the gene) indicates the likelihood of successful treatment is higher than if the RecQ helicase family gene is expressed at a higher level (because the gene is unmethylated).

The likelihood of successful treatment may also be considered higher according to this method if there are higher levels of methylation and/or lower levels of expression of the RecQ helicase family gene than in normal (i.e. non-cancerous) tissues. These methods are referred to hereinafter as the “pharmacogenetic” methods of the invention.

Preferably, the method is used for predicting the likelihood of successful treatment of cancer with a topoisomerase inhibitor and/or a DNA damaging agent.

For the avoidance of doubt it is confirmed that the invention also provides a method for predicting the likelihood of successful treatment of cancer with a topoisomerase inhibitor and/or a DNA damaging agent and/or a DNA methyltransferase inhibitor and/or a HDAC inhibitor comprising:

(a) determining the methylation status of a RecQ helicase family gene, in particular the WRN gene, in a sample obtained from a subject, wherein if the RecQ helicase family gene is unmethylated the likelihood of successful treatment is lower than if the RecQ helicase family gene is methylated; and/or
(b) determining the expression levels of a RecQ helicase family gene in a sample obtained from a subject, wherein a normal level of expression of the RecQ helicase family gene (due to a lack of methylation of the gene) indicates the likelihood of successful treatment is lower than if the RecQ helicase family gene is expressed at a lower level (because the gene is methylated). This method is based around the observations presented herein for the first time in which a lack of methylation of the WRN gene leading to decreased WRN levels provides resistance against certain anti-cancer agents. Thus, the method may be considered, in the alternative, a method for predicting the likelihood of resistance to treatment of cancer with a topoisomerase inhibitor and/or a DNA damaging agent and/or a DNA methyltransferase inhibitor and/or a HDAC inhibitor comprising:
(a) determining the methylation status of a RecQ helicase family gene, in particular the WRN gene, in a sample obtained from a subject, wherein if the RecQ helicase family gene is unmethylated the likelihood of resistance to treatment is higher than if the RecQ helicase family gene is methylated; and/or
(b) determining the expression levels of a RecQ helicase family gene in a sample obtained from a subject, wherein a normal level of expression of the RecQ helicase family gene (due to a lack of methylation of the gene) indicates the likelihood of resistance to treatment is higher than if the RecQ helicase family gene is expressed at a lower level (because the gene is methylated).

Preferably, the method is used for predicting the likelihood of resistance to treatment of cancer with a topoisomerase inhibitor and/or a DNA damaging agent.

Furthermore, the invention provides a method of selecting a suitable treatment regimen for cancer comprising:

(a) determining the methylation status of a RecQ helicase family gene, and preferably the WRN gene, in a sample obtained from a subject, wherein if the RecQ helicase family gene, which is preferably the WRN gene, is hypermethylated a topoisomerase inhibitor and/or a DNA damaging agent and/or a DNA methyltransferase inhibitor and/or a HDAC inhibitor is selected for treatment; and/or
(b) determining the expression levels of a RecQ helicase family gene in a sample obtained from a subject, wherein a reduced level of expression of the RecQ helicase family gene (as caused by increased methylation of the gene) indicates a topoisomerase inhibitor and/or a DNA damaging agent and/or a DNA methyltransferase inhibitor and/or a HDAC inhibitor should be selected for treatment. These methods are referred to hereinafter as the “treatment regimen” methods of the invention. The treatment regimen may thus be chosen according to these methods if there are higher levels of methylation and/or lower levels of expression detected than in normal (i.e. non-cancerous) tissues.

Preferably, the method is used for selecting a suitable treatment regimen for cancer in which treatment with a topoisomerase inhibitor and/or a DNA damaging agent is selected if the RecQ helicase family gene, preferably the WRN gene, is methylated which may be determined by determining expression levels as discussed above.

Also, the invention provides in a further aspect a method of selecting a suitable treatment regimen for cancer comprising:

(a) determining the methylation status of a RecQ helicase family gene, preferably the WRN gene, in a sample obtained from a subject, wherein if the RecQ helicase family gene, and preferably the WRN gene, is unmethylated treatment using a topoisomerase inhibitor and/or a DNA damaging agent and/or a DNA methyltransferase inhibitor and/or a HDAC inhibitor is contra-indicated; and/or
(b) determining the expression levels of a RecQ helicase family gene in a sample obtained from a subject, wherein a normal level of expression of the RecQ helicase family gene (due to a lack of methylation of the gene) indicates treatment using a topoisomerase inhibitor and/or a DNA damaging agent and/or a DNA methyltransferase inhibitor and/or a HDAC inhibitor is contra-indicated. Preferably, the method is used for selecting a suitable treatment regimen for cancer in which treatment with a topoisomerase inhibitor and/or a DNA damaging agent is contra-indicated if the RecQ helicase family gene, in particular the WRN gene, is unmethylated or where expression of the RecQ helicase family gene, in particular the WRN gene, is normal (i.e. not decreased due to the lack of methylation of the gene).

In a still further aspect, the invention provides a method of diagnosing cancer comprising:

(a) determining the methylation status of a RecQ helicase family gene, and preferably the WRN gene, in a sample obtained from a subject, wherein hypermethylation of the RecQ helicase family gene, in particular the WRN gene, is indicative of cancer; and/or
(b) determining the expression levels of a RecQ helicase family gene, in particular the WRN gene, in a sample obtained from a subject, wherein a reduced level of expression of the RecQ helicase family gene, in particular the WRN gene is indicative of cancer.

These methods are referred to hereinafter as the “diagnostic” methods of the invention. The diagnosis of cancer may thus be made according to this method if there are higher levels of methylation than in normal (i.e. non-cancerous) tissues.

All methods of the invention may be carried out with respect to any member of the RecQ helicase family, as discussed above. Preferred examples include RecQL, BLM (BLOOM), WRN, RecQ4 and RecQ5. Particularly preferred is the WRN gene which is shown herein for the first time to be methylated and thus down-regulated in various cancer types.

All of these methods are most preferably in vitro methods carried out on an isolated sample. In one embodiment, the methods may also include the step of obtaining the sample.

In a most preferred embodiment, the subject is a human subject. Generally the subject may be a patient wherein a potential cancer has been identified and the method may be used to determine if indeed there is a potentially dangerous lesion and also to guide treatment depending upon the methylation status of the RecQ helicase family gene, WRN gene.

The test sample is generally any sample taken preferably from the subject under test in which the methylation status of the RecQ helicase family gene is reflective of the cancer status. The sample is most preferably a tissue sample taken from the subject which is suspected of being a tumor. The tissue chosen may be determined by the type of cancer which is suspected or is to be treated. Preferred cancer types according to the methods of the invention are discussed in more detail below and the skilled person would immediately appreciate which type of sample would be appropriate depending upon the cancer concerned.

However, any other suitable test sample in which methylation status of a RecQ helicase family, not preferably the WRN gene, can be determined to indicate the presence of cancer or the likelihood of successful treatment of cancer with the specified agents are included within the scope of the invention. Test samples for diagnostic, prognostic, or personalised medicinal uses may be obtained from surgical samples, such as biopsies or fine needle aspirates, from paraffin embedded tissues or from a body fluid for example. Non-limiting examples include whole blood, serum, plasma, urine, chyle, stool, ejaculate, sputum, nipple aspirate and saliva.

All of these aspects of the invention rely upon determining the methylation status of a RecQ helicase family gene and/or determining expression levels which are directly related to methylation status of the gene. Preferably, the WRN gene is assessed.

### Determining Gene Expression Levels

The decreased level of expression of a RecQ helicase family gene may, as necessary, be measured in order to determine if it is statistically significant in the sample. This helps to provide a reliable test for the methods of the invention. Any method for determining whether the expression level of a RecQ helicase family gene is significantly reduced may be utilised. Such methods are well known in the art and routinely employed. For example, statistical analyses may be performed using an analysis of variance test. Typical P values for use in such a method would be P values of <0.05 or 0.01 or 0.001 when determining whether the relative expression or activity is statistically significant. A change in expression may be deemed significant if there is at least a 10% decrease for example. The test may be made more selective by making the change at least 15%, 20%, 25%, 30%, 35%, 40% or 50%, for example, in order to be considered statistically significant.

In a preferred embodiment, the decreased level of expression or activity of a RecQ helicase family gene is determined with reference to a control sample. This control sample is preferably taken from normal (i.e. non tumorigenic) tissue in the subject, where expression of the RecQ helicase family gene is normal. Additionally or alternatively control samples may also be utilised in which there is known to be a lack of expression of the RecQ helicase family gene.

Suitable additional controls may also be included to ensure that the test is working properly, such as measuring levels of expression or activity of a suitable reference gene in both test and control samples.

In a most preferred embodiment, the subject is a human subject. Generally, the subject will be a patient wherein cancer is suspected or a potential cancer has been identified and the method may be used to determine if indeed there is a cancer present. The methods of the invention may be used in conjunction with known methods for detecting cancer.

In one preferred embodiment, the method of the invention is carried out by determining protein expression of a RecQ helicase family gene. In a most preferred embodiment, total loss of protein expression of the RecQ helicase is observed in the sample in order to conclude a diagnosis of cancer, or to make a decision on the best course of treatment in accordance with the other methods of the invention. However, partial loss of RecQ helicase expression may also be relevant, due to methylation of the relevant gene.

Levels of protein expression may be determined by a number of techniques, as are well known to one of skill in the art. Examples include western blots, immunohistochemical staining and immunolocalization, immunofluorescene, enzyme-linked immunosorbent assay (ELISA), immunoprecipitation assays, agglutination reactions, radioimmunoassay, flow cytometry and equilibrium dialysis. These methods generally depend upon a reagent specific for identification of the RecQ family helicase. The reagent is preferably an antibody and may comprise monoclonal or polyclonal antibodies. Fragments and derivatized antibodies may also be utilised, to include without limitation Fab fragments, ScFv, single domain antibodies, nanoantibodies, heavy chain antibodies etc which retain RecQ helicase binding function. Any detection method may be employed in accordance with the invention. The nature of the reagent is not limited except that it must be capable of specifically identifying the appropriate RecQ helicase family member.

Of course, in the case of a positive diagnosis of cancer, there will be reduced levels of the relevant RecQ helicase, and perhaps no RecQ helicase at all. In one embodiment this will present a negative result, if the RecQ helicase specific reagent is one which binds to the wild type or full length protein. In this case, use of suitable controls ensures that false diagnoses will not be made, for example caused by degraded or non-specific reagents. Thus, the same reagent can be tested on samples in which it is known that the RecQ helicase family member is expressed. A positive result in this control sample, combined with a negative result in the test sample provides a confident diagnosis of cancer and removes any doubt over the quality of the reagent.

RecQ helicase family gene expression may also be monitored at the RNA level in one embodiment. Thus a decreased or abolished level of the RecQ helicase family gene expression, caused by methylation of the gene, results in lower levels of functional RecQ helicase protein and this is indicative of cancer and/or likelihood of successful treatment and/or directs the course of treatment.

Suitable methods for determining expression of a RecQ helicase family gene at the RNA level are well known in the art. Methods employing nucleic acid probe hybridization to the relevant RecQ helicase transcript may be employed for measuring the presence and/or level of RecQ helicase mRNA. Such methods include use of nucleic acid probe arrays (microarray technology) and Northern blots. Advances in genomic technologies now permit the simultaneous analysis of thousands of genes, although many are based on the same concept of specific probe-target hybridization.

Sequencing-based methods are an alternative. These methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest.

In one embodiment, the levels of RecQ helicase gene expression are determined using reverse transcriptase polymerase chain reaction (RT-PCR). RT-PCR is a well known technique in the art which relies upon the enzyme reverse transcriptase to reverse transcribe mRNA to form cDNA, which can then be amplified in a standard PCR reaction. Protocols and kits for carrying out RT-PCR are extremely well known to those of skill in the art and are commercially available.

In a preferred embodiment, the RT-PCR is carried out in real time and in a quantitative manner. Real time quantitative RT-PCR has been thoroughly described in the literature (see Gibson et al for an early example of the technique) and a variety of techniques are possible. Examples include use of Taqman, Molecular Beacons, LightCycler (Roche), Scorpion and Amplifluour systems. All of these systems are commercially available and well characterised, and may allow multiplexing (that is, the determination of expression of multiple genes in a single sample).

These techniques produce a fluorescent read-out that can be continuously monitored. Real-time techniques are advantageous because they keep the reaction in a “single tube”. This means there is no need for downstream analysis in order to obtain results, leading to more rapidly obtained results. Furthermore, keeping the reaction in a “single tube” environment reduces the risk of cross contamination and allows a quantitative output from the methods of the invention. This may be particularly important in the clinical setting of the present invention.

It should be noted that whilst PCR is a preferred amplification method, to include variants on the basic technique such as nested PCR, equivalents may also be included within the scope of the invention. Examples include isothermal amplification techniques such as NASBA, 3SR, TMA and triamplification, all of which are well known in the art and commercially available. Other suitable amplification methods include the ligase chain reaction (LCR) (Barringer et al, 1990), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995) and nick displacement amplification (WO2004/067726).

### Determining Methylation Status

In one embodiment, the methylation status of the promoter region of a RecQ helicase family gene is determined. In particular, the relevant CpG islands may be assessed in accordance with the methods of the invention.

As discussed in the experimental section below, CpG islands are present in the WRN gene. Preferably, the methylation status of these regions is analysed to determine the methylation status. In a most preferred embodiment, the promoter region of the WRN gene is assessed to determine its methylation status. Preferably, the CpG island positioned around the transcription start site of the WRN gene is analysed to determine its methylation status.

In one embodiment, the region of the WRN gene comprising, consisting essentially of, or consisting of the nucleotide sequence set forth as SEQ ID NO: 1 is analysed in order to determine its methylation status.

It is noted that the methylation status of additional genes may also be determined in order to supplement the methods of the invention.

In one preferred embodiment of these aspects of the invention (i.e. the pharmacogenetic, treatment regimen and diagnostic methods) the methylation status of the RecQ helicase family gene, in particular the WRN gene, (or portion thereof, especially the CpG islands) is determined using methylation specific PCR (MSP). However, any technique may be utilised to determine the methylation status of the RecQ helicase family gene, which is most preferably WRN. Examples include the well-known techniques of COBRA (which relies upon use of restriction enzymes to reveal methylation dependent sequence differences in PCR products of sodium bisulfite—treated DNA), bisulphite sequencing and use of arrays which can distinguish between bisulphite treated nucleic acid molecules following amplification. A review of some useful techniques is provided in Nucleic acids research, 1998, Vol. 26, No. 10, 2255-2264, which reference is incorporated herein in its entirety. DNA methylation analysis may also take advantage of the sensitivity of restriction enzymes to the methylation status of the DNA. DNA methylation analysis has been performed successfully with a number of techniques which include MALDI-TOF, MassARRAY, MethyLight, Quantitative analysis of ethylated alleles (QAMA), enzymatic regional methylation assay (ERMA), HeavyMethyl, QBSUPT, MS-SNuPE, MethylQuant, Quantitative PCR sequencing and oligonucleotide-based microarray systems.

Additional methods for the identification of methylated CpG dinucleotides utilize the ability of the methyl binding domain (MBD) of the McCP2 protein to selectively bind to methylated DNA sequences (Cross et al, 1994; Shiraishi et al, 1999). Restriction endonuclease digested genomic DNA is loaded onto expressed His-tagged methyl-CpG binding domain that is immobilized to a solid matrix and used for preparative column chromatography to isolate highly methylated DNA sequences.

The MSP technique will be familiar to one of skill in the art. In the MSP approach, DNA may be amplified using primer pairs designed to distinguish methylated from unmethylated DNA by taking advantage of sequence differences as a result of sodium-bisulfite treatment (Herman J G, Graff J R, Myohanen S, Nelkin B D, Baylin S B. Methylation-specific PCR: a novel PCR assay for methylation status of CpG islands. Proc Natl Acad Sci USA 1996; 93(18):9821-9826 and see WO 97/46705, incorporated herein by reference). After sodium-bisulfite treatment unmethylated cytosines are converted to uracil whereas methylated cytosines remain unconverted.

A specific example of the MSP technique is designated real-time quantitative MSP (QMSP), which permits reliable quantification of methylated DNA in real time. These methods are generally based on the continuous optical monitoring of an amplification procedure and utilise fluorescently labelled primers and/or probes. They represent a specific application of the well known and commercially available real-time amplification techniques such as TAQMAN®, MOLECULAR BEACONS®, LIGHTCYCLER®, AMPLIFLUOUR® and SCORPION® etc as described in more detail herein. Often, these real-time methods are used with the polymerase chain reaction (PCR). Real-time methods do not need to be utilised, however. Amplification products may simply be run on a suitable gel, such as an agarose gel, to determine if the expected sized products are present. This may involve use of ethidium bromide staining and visualisation of the DNA bands under a UV illuminator for example.

In real-time embodiments, methylation status may be determined by using the ratio between the signal of the marker under investigation and the signal of a reference gene where methylation status is known (such as β-actin for example), or by using the ratio between the methylated marker and the sum of the methylated and the non-methylated marker.

In one embodiment, each clinical sample is measured in duplicate and for both Cₜ values (cycles at which the amplification curves crossed the threshold value, set automatically by the relevant software) copy numbers are calculated. The average of both copy numbers (for each gene) is used for the result classification. To quantify the final results for each sample two standard curves are used, one for either the reference gene (β-actin or the non-methylated marker) and one for the methylated version of the marker. The results of all clinical samples (when m-Gene was detectable) are expressed as 1000 times the ratio of “copies m-Gene”/“copies β-actin” or “copies m-Gene”/“copies u-Gene+m-Gene” and then classified accordingly (methylated, non-methylated or invalid) (u=unmethylated; m=methylated).

In one embodiment, primers useful in MSP carried out on the promoter region of the WRN gene are provided. These primers comprise, consist essentially of or consist of the following sequences:

### Unmethylated WRN Specific Primers:

| | | |
|---|---|---|
| SEQ ID NO: 2 | 5′-GTA GTT GGG TAG GGG TAT TGT TTG T-3′ | (sense) |
| SEQ ID NO: 3 | 5′-AAA CAA AAT CCA CCA CCC ACC CC-3′ | (antisense) |

### Methylated WRN Specific Primers:

| | | |
|---|---|---|
| SEQ ID NO: 4 | 5′-CGG GTA GGG GTA TCG TTC GC-3′ | (sense) |
| SEQ ID NO: 5 | 5′-AAC GAA ATC CAC CGC CCG CC-3′ | (antisense). |

These primers are located −36 (sense) and +129 (antisense) from the transcription start site.

In a further embodiment, bisulphite sequencing is utilised in order to determine the methylation status of the RecQ helicase family gene, preferably the WRN gene. Primers may be designed for use in sequencing through the important CpG islands in the RecQ helicase family gene. Thus, primers may be designed in both the sense and antisense orientation to direct sequencing across the promoter region of the relevant RecQ helicase family gene. In one embodiment, in which the WRN gene is sequenced, bisulphite sequencing may be carried out by using sequencing primers which comprise, consist essentially of or consist of the following sequences, and which may be used in isolation or in combination to sequence both strands:

| | |
|---|---|
| SEQ ID NO: 6 | 5′-AGG TTT TTA GTY GGY GGG TAT TTA-3′(sense) |

wherein “Y” represents a pyrimidine nucleotide

| | |
|---|---|
| SEQ ID NO: 7 | 5′-AAC CCC CTC TTC CCC TCA-3′(antisense) |

These sequencing primers form a further aspect of the invention.

Other nucleic acid amplification techniques, in addition to PCR (which includes real-time versions thereof and variants such as nested PCR), may also be utilised, as appropriate, to detect the methylation status of the RecQ helicase family gene. Such amplification techniques are well known in the art, and include methods such as NASBA (Compton, 1991, 3SR (Fahy et al., 1991) and Transcription Mediated Amplification (TMA). Other suitable amplification methods include the ligase chain reaction (LCR) (Barringer et al, 1990), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO 90/06995) and nick displacement amplification (WO 2004/067726). This list is not intended to be exhaustive; any nucleic acid amplification technique may be used provided the appropriate nucleic acid product is specifically amplified. Thus, these amplification techniques may be tied in to MSP and/or bisulphite sequencing techniques for example.

Sequence variation that reflects the methylation status at CpG dinucleotides in the original genomic DNA offers two approaches to primer design. Firstly, primers may be designed that themselves do not cover any potential sites of DNA methylation. Sequence variation at sites of differential methylation are located between the two primers. Such primers are used in bisulphite genomic sequencing, COBRA and Ms-SnuPE for example. Secondly, primers may be designed that anneal specifically with either the methylated or unmethylated version of the converted sequence. If there is a sufficient region of complementarity, e.g., 12, 15, 18, or 20 nucleotides, to the target, then the primer may also contain additional nucleotide residues that do not interfere with hybridization but may be useful for other manipulations. Examples of such other residues may be sites for restriction endonuclease cleavage, for ligand binding or for factor binding or linkers or repeats. The oligonucleotide primers may or may not be such that they are specific for modified methylated residues.

One way to distinguish between modified and unmodified DNA is to hybridize oligonucleotide primers which specifically bind to one form or the other of the DNA. After hybridization, an amplification reaction can be performed and amplification products assayed. The presence of an amplification product indicates that a sample hybridized to the primer. The specificity of the primer indicates whether the DNA had been modified or not, which in turn indicates whether the DNA had been methylated or not.

Another way to distinguish between modified and unmodified DNA is to use oligonucleotide probes which may also be specific for certain products. Such probes may be hybridized directly to modified DNA or to amplification products of modified DNA. Oligonucleotide probes can be labelled using any detection system known in the art. These include but are not limited to fluorescent moieties, radioisotope labelled moieties, bioluminescent moieties, luminescent moieties, chemiluminescent moieties, enzymes, substrates, receptors, or ligands.

In the MSP technique, amplification is achieved with the use of primers specific for the sequence of the gene whose methylation status is to be assessed. In order to provide specificity for the nucleic acid molecules, primer binding sites corresponding to a suitable region of the sequence may be selected. The skilled reader will appreciate that the nucleic acid molecules may also include sequences other than primer binding sites which are required for detection of the methylation status of the gene, for example RNA Polymerase binding sites or promoter sequences may be required for isothermal amplification technologies, such as NASBA, 3SR and TMA.

TMA (Gen-probe Inc.) is an RNA transcription amplification system using two enzymes to drive the reaction, namely RNA polymerase and reverse transcriptase. The TMA reaction is isothermal and can amplify either DNA or RNA to produce RNA amplified end products. TMA may be combined with Gen-probe's Hybridization Protection Assay (HPA) detection technique to allow detection of products in a single tube. Such single tube detection is a preferred method for carrying out the invention.

As mentioned above, in a preferred embodiment, the methylation status of the RecQ helicase family gene, in particular the WRN gene, is determined by methylation specific PCR, preferably real-time methylation specific PCR. In specific embodiments, the real-time methylation specific PCR comprises use of TAQMAN probes and/or MOLECULAR BEACONS probes and/or AMPLIFLUOUR primers and/or LIGHT-CYCLER and/or FRET probes and/or SCORPION primers.

Since the WRN promoter appears to be completely unmethylated in normal tissues, it is clear that the methods of the invention are particularly useful, since the detection of methylation in this region is readily observable as being significant in terms of a cancer diagnosis and also in selecting suitable treatment regimens and for determining the likelihood of successful treatment or resistance to treatment with certain anti-cancer agents. Likewise the detection of unmethylated WRN genes is also of relevance.

However, when determining methylation status, it may still be beneficial to include suitable controls in order to ensure the method chosen to assess this parameter is working correctly and reliably. For example, suitable controls may include assessing the methylation status of a gene known to be methylated. This experiment acts as a positive control to ensure that false negative results are not obtained (i.e. a conclusion of a lack of methylation is made even though the WRN gene or other RecQ helicase family gene may, in fact, be methylated). The gene may be one which is known to be methylated in the sample under investigation or it may have been artificially methylated, for example by using a suitable methyltransferase enzyme, such as SssI methyltransferase. In one embodiment, the RecQ helicase family gene, preferably the WRN gene, may be assessed in normal lymphocytes, following treatment with SssI methyltransferase, as a positive control.

Additionally or alternatively, suitable negative controls may be employed with the methods of the invention. Here, suitable controls may include assessing the methylation status of a gene known to be unmethylated. This experiment acts as a negative control to ensure that false positive results are not obtained (i.e. a conclusion of methylation is made even though the RecQ helicase family gene may, in fact, be unmethylated). The gene may be one which is known to be unmethylated in the sample under investigation or it may have been artificially demethylated, for example by using a suitable DNA methyltransferase inhibitor, such as those discussed in more detail below. In one embodiment, the RecQ helicase family gene, in particular the WRN gene, may be assessed in normal lymphocytes as a negative control, since it has been shown for the first time herein that the WRN gene is completely unmethylated in normal tissues.

As mentioned above, in a further aspect the invention provides a method of treating cancer in a subject comprising administration of a topoisomerase inhibitor and/or a DNA damaging agent and/or a DNA methyltransferase inhibitor and/or a HDAC inhibitor, wherein the subject has been selected for treatment on the basis of:

(a) determining the methylation status of a RecQ helicase family gene, in particular the WRN gene, according to the methods of the invention; and/or
(b) measuring the expression levels of a RecQ helicase family gene, in particular the WRN gene according to any of the methods of the invention.

Thus, for the patient population where the RecQ helicase family gene is methylated, which leads to decreased gene expression, this type of treatment is recommended. Preferably, treatment involves use of a topoisomerase inhibitor and/or a DNA damaging agent. This method is referred to hereinafter as the “method of treatment” aspect of the invention.

In a related aspect, the invention also provides for the use of a topoisomerase inhibitor and/or a DNA damaging agent and/or a DNA methyltransferase inhibitor and/or a HDAC inhibitor in the manufacture of a medicament for use in treating cancer in a subject, wherein the subject has been selected for treatment on the basis of:

(a) determining the methylation status of a RecQ helicase family gene, in particular the WRN gene, according to the methods of the invention; and/or
(b) measuring the expression levels of a RecQ helicase family gene, in particular the WRN gene according to any of the methods of the invention.

Thus, the patient population may be selected for treatment on the basis of their methylation status with respect to the relevant RecQ helicase family gene, in particular WRN which leads to down regulation of gene expression of the corresponding gene. This leads to a much more focussed and personalised form of medicine and thus leads to improved success rates since patients will be treated with drugs which are most likely to be effective. In the experimental section below, it is shown for the first time that methylation status at the WRN gene is useful for indicating whether certain treatments such as use of topoisomerase inhibitors is likely to prove successful.

In a related aspect the invention also provides a method for treating cancer in a subject, said subject having a reduced level or activity of a RecQ helicase, in particular WRN, comprising reconstitution of RecQ helicase activity, in particular WRN activity, in the subject. Hereinafter, this aspect of the invention is referred to as the “gene therapy” aspect of the invention.

In a related aspect, the invention provides for the use of a vector carrying a RecQ helicase family gene, and preferably the WRN gene, in the manufacture of a medicament for treating cancer in a subject, wherein the subject has been selected for treatment according to the methods of the invention. Methylation of the RecQ helicase family gene, preferably the WRN gene, selects the subject for treatment. The methylation may be determined at the level of gene expression.

For the avoidance of doubt, when reference is made to the “method of treatment” and “gene therapy” methods, reference is intended to also be made to the uses described above.

Thus, methylation of RecQ helicase family genes, in particular the WRN gene, has been shown for the first time herein to be relevant to cancer. Methylation is linked to down regulation of RecQ helicase family gene expression. Accordingly, it is predicted that methods for increasing RecQ helicase family gene expression will result in improved recovery from cancer in cases where the RecQ helicase family gene is methylated. As shown in the experimental section below, reconstitution of functional WRN produces tumour suppressor like features in transfected cells.

In a preferred embodiment, the reduced level or activity of the RecQ helicase, in particular WRN, is detected by determining the methylation status of the RecQ helicase family gene. This may be done according to any of the methods of the invention described above (the pharmacogenetic, treatment regimen and diagnostic methods). Thus, a particular subgroup of subjects suffering from, or predicted to have a likelihood of developing, cancer is selected for treatment according to whether the RecQ helicase family gene, in particular the WRN gene, is methylated or not (which may be determined at the level of gene expression if desired).

For all of the above described methods of the invention (the pharmacogenetic, treatment regimen, diagnostic, method of treatment and gene therapy methods) the cancer may be any cancer in which methylation of a RecQ helicase family gene, preferably the WRN gene, is relevant. However in a preferred embodiment, the cancer is selected from epithelial tumours, mesenchymal tumours and haematological malignancies.

Particular cancer types which are relevant in accordance with the present invention include those selected from colorectal cancer, non-small cell lung cancer, gastric cancer, prostate cancer, breast cancer, ovarian cancer and thyroid cancer. As is shown in the experimental section below, particularly relevant cancer types within the scope of the present invention include colorectal or ovarian cancer. Any of these cancers may comprise epithelial tumours.

In one embodiment, the mesenchymal tumour is selected from chondrosarcomas and osteosarcomas. These two types of mesenchymal tumour have both been shown to have a link with WRN methylation in the present invention.

In a further embodiment, the haematological malignancy is selected from non-Hodgkin lymphoma, acute lymphoblastic leukaemia and acute myeloblastic leukaemia. The link between WRN methylation and non-Hodgkin lymphoma has been shown to be most strong and so this particular type of lymphoma is preferred in the methods of the invention.

For all of the (pharmacogenetic methods, treatment regimen methods and methods of treatment) of the invention, the topoisomerase inhibitor may be any suitable inhibitor of topoisomerase which is suitable for treating cancer in the presence of methylation of a RecQ helicase family gene, and preferably the WRN gene.

In one preferred embodiment, the topoisomerase inhibitor comprises, consists essentially of or consists of a topoisomerase I inhibitor (topo I inhibitor). Clinical trials are underway in respect of a number of topo I inhibitors. Most of these inhibitors are derived from the plant extract camptothecin (Ewewuedo & Ratain, The Oncologist (1997) 2:359-364). Thus, in a preferred embodiment, the topoisomerase I inhibitor comprises camptothecin, or a derivative thereof.

By derivative is meant any altered version of camptothecin which retains suitable topo I inhibitor activity. Preferred derivative for the purposes of the present invention comprise, consist essentially of or consist of irinotecan (CPT-11), topotecan, lurtotecan and/or exatecan.

For all of the relevant methods (pharmacogenetic method, treatment regimen methods and methods of treatment) of the invention, the DNA damaging agent may be any suitable DNA damaging agent which is suitable for treating cancer in the presence of methylation of a RecQ helicase family gene, preferably the WRN gene. As is shown in the experimental section below, methylation of the WRN gene makes cancer cells especially susceptible to treatment using DNA damaging agents.

Accordingly, in one embodiment, the DNA damaging agent comprises, consists essentially of or consists of any of a DNA interstrand cross linker, UV light, gamma irradiation and tritiated thymidine.

The UV light may be of any suitable wavelength to cause damage to DNA in the cancer cells in which the RecQ helicase family gene, and preferably the WRN gene is methylated. Typically, the UV light will be of short wavelength, preferably in the UV-B or UV-C region.

The DNA interstrand cross linker may comprise any suitable cross-linker which can be used to destroy cancer cells. Preferred examples include, but are not limited to, any of mitomycin C, cis-platinum, 3,6-diaziridinyl-2,5-bis(carboethoxyamino)-1,4-benzoquinone (diaziquone, AZQ), melphalan and/or chlorambucil.

For all of the relevant methods (pharmacogenetic methods, treatment regimen methods and methods of treatment) of the invention, the DNA methyltransferase inhibitor may be any suitable inhibitor of DNA methyltransferase which is suitable for treating cancer in the presence of methylation of the RecQ helicase family gene, in particular the WRN gene. As is shown in the experimental section below, methylation of the WRN gene is linked to cancer and so preventing this methylation is predicted to help to treat cancer.

The DNA methyltransferase inhibitor may, in one embodiment, be one which reduces expression of DNMT genes, such as suitable antisense molecules, or siRNA molecules which mediate RNAi for example. The design of a suitable siRNA molecule is within the capability of the skilled person and suitable molecules can be made to order by commercial entities (see for example, www.ambion.com). Preferably, the DNA methyltransferase gene is (human) DNMT1.

Alternatively, the agent may be a direct inhibitor of DNMTs. Examples include modified nucleotides such as phosphorothioate modified oligonucleotides (FIG. 6 of Villar-Garea, A. And Esteller, M. DNA demethylating agents and chromatin-remodelling drugs: which, how and why? Current Drug Metabolism, 2003, 4, 11-31) and nucleosides and nucleotides such as cytidine analogues. Suitable examples of cytidine analogues include 5-azacytidine, 5-aza-2′-deoxycytidine, 5-fluouro-2′-deoxycytidine, pseudoisocytidine, 5,6-dihydro-5-azacytidine, 1-β-D-arabinofuranosyl-5-azacytosine (known as fazabarine) (see FIG. 4 of Villar-Garea, A. And Esteller, M. DNA demethylating agents and chromatin-remodelling drugs: which, how and why? Current Drug Metabolism, 2003, 4, 11-31).

In another embodiment, the DNA methyltransferase inhibitor comprises Decitabine. Full details of this drug can be found at www.supergen.com for example.

Additional DNMT inhibitors include S-Adenosyl-Methionine (SAM) related compounds like ethyl group donors such as L-ethionine and non-alkylating agents such as S-adenosyl-homocysteine (SAH), sinefungin, (S)-6-methyl-6-deaminosine fungin, 6-deaminosinefungin, N4-adenosyl-N4-methyl-2,4-diaminobutanoic acid, 5′-methylthio-5′-deoxyadenosine (MTA) and 5′-amino-5′-deoxyadenosine (Villar-Garea, A. And Esteller, M. DNA demethylating agents and chromatin-remodelling drugs: which, how and why? Current Drug Metabolism, 2003, 4, 11-31).

Further agents which may alter DNA methylation and which may, therefore, be useful in the present compositions include organohalogenated compounds such as chloroform etc, procianamide, intercalating agents such as mitomycin C, 4-aminobiphenyl etc, inorganic salts of arsenic and selenium and antibiotics such as kanamycin, hygromycin and cefotaxim (Villar-Garea, A. And Esteller, M. DNA demethylating agents and chromatin-remodelling drugs: which, how and why? Current Drug Metabolism, 2003, 4, 11-31).

However, any suitable DNA methyltransferase inhibitor which is capable of increasing the expression of a RecQ helicase family gene, and preferably the WNR gene, and thus can contribute to the treatment of cancer, is included within the scope of the invention.

Particularly preferred DNMT inhibitors in the present invention comprise, consists essentially of or consists of 5-azacytidine and/or zebulaine.

For all of the pharmacogenetic, treatment regimen and methods of treatment of the invention, the histone deacetylase (HDAC) inhibitor may be any suitable inhibitor of HDAC activity which is suitable for treating cancer in the presence of methylation of a RecQ helicase family gene, and preferably the WRN gene.

In a preferred embodiment, the histone deacetylase (HDAC) inhibitor comprises at least one of trichostatin A (TSA), suberoyl hydroxamic acid (SBHA), 6-(3-chlorophenylureido)caproic hydroxamic acid (3-Cl-UCHA), m-carboxycinnamic acid bishydroxylamide (CBHA), suberoylanilide hydroxamic acid (SAHA), azelaic bishydroxamic acid (ABHA), pyroxamide, scriptaid, aromatic sulfonamides bearing a hydroxamic acid group, oxamflatin, trapoxin, cyclic-hydroxamic-acid containing peptides, FR901228, MS-275, MGCD0103 (see www.methylgene.com), short-chain fatty acids and N-acetyldinaline (Villar-Garea, A. And Esteller, M. DNA demethylating agents and chromatin-remodelling drugs: which, how and why? Current Drug Metabolism, 2003, 4, 11-31).

With respect to the gene therapy methods (and uses) set out above, reconstituting RecQ helicase activity, and preferably WRN activity, preferably comprises delivery of wild type copies of the RecQ helicase family gene, and preferably the WRN gene, into the subject. However, any functional version may be utilised provided the desired therapeutic effect is achieved.

Any suitable vector for delivery of functional copies of the RecQ helicase family gene, in particular the WRN gene, may be utilised according to the method of the invention. One principal requirement is that tissue specificity of delivery and expression is achieved. The two major sources of vectors which may be utilised comprise viral vectors and non-viral vectors.

Within the group of viral vectors, preferred types include adenoviruses, retroviruses, in particular Moloney murine leukaemia virus (Mo-MLV), adeno-related viruses and herpes simplex virus type I. Typically, the gene of interest, in this case encoding a RecQ helicase and preferably WRN, will be included in the viral genome, preferably in the “non-essential” region of the viral genome. In addition, it is important to remove virally encoded proto-oncogenes from the viral vector genome. The virus may be made replication incompetent to prevent unwanted replication once the virus has been targeted.

In terms of targeting the viral vector to the desired site, a number of possibilities exist. For example, the env gene (which encodes the viral vector's envelope) may be engineered or replaced with the env gene from a different virus to alter the range of cells the viral vector will “infect”. Furthermore, alteration of the viral tropism may be achieved by using suitable antibodies raised against antigenic determinants on the cell surface of the desired target cells. The antibodies, which include all derivatives thereof, such as scFV, nanobodies, VH domains, Fab fragments etc., may be genetically incorporated into the viral vectors to provide targeted gene delivery of the WRN gene. Most preferred is use of scFV (Hedley et al., Gene Therapy (2006) 13, 88-94). The viral vectors may have many genes removed, such as packaging genes, in order to reduce immunogenicity and/or infectivity. These functions may thus be supplied by a helper virus.

Due to their high efficiency of integration, low pathogenicity and high efficacy, adenoviruses are a preferred vector according to the methods of the invention.

Alternatives to viral vectors include direct gene delivery, use of other delivery agents and use of molecular conjugates. Tissue specific promoters may be employed as appropriate. Direct gene delivery may be achieved for example by microinjection of a suitable vector, such as a plasmid carrying a RecQ helicase family gene, in particular the WRN gene, directly into the tissue of interest. Alternatives include use of ballistic transformation, for example using vector coated onto suitable particles (e.g. gold particles). Additional delivery agents include liposomes and derivatives thereof. As discussed above, targeting proteins such as antibodies and derivatives thereof may be utilised in order to ensure delivery to the cells of interest. Molecular conjugates may include suitable proteins conjugated to the DNA of interest using a suitable DNA binding agent.

The methods of treatment and medical uses according to the gene therapy aspects of the invention may incorporate any and all of the preferred aspects described in respect of the other methods of the invention (diagnostic, pharmacogenetic and treatment regimen methods and also methods of treating cancer) as described above. Preferably, the diagnostic methods and/or the pharmacogenetic methods and/or the treatment regimen methods of the invention are carried out as a prelude to, or as an integral part of the methods of treating cancer according to the gene therapy aspects of the invention. The gene therapy methods may be synergistically combined with those of the methods of treatment according to the invention.

Thus, for example, the description of suitable methods for determining methylation levels of a RecQ helicase family gene and preferably WRN, suitable test samples, preferred subjects and specific types of cancer which may be treated all apply mutatis mutandis to these aspects of the invention and are not repeated here simply for reasons of conciseness.

### Kits

The invention also provides kits which may be used in order to carry out the methods of the invention. The kits may incorporate any of the preferred features mentioned in connection with the various methods (and uses) of the invention above.

Thus, a kit is provided for:
(a) predicting the likelihood of successful treatment of cancer and/or the likelihood of resistance to treatment of cancer with a topoisomerase inhibitor and/or a DNA damaging agent and/or a DNA methyltransferase inhibitor and/or a HDAC inhibitor, and/or
(b) selecting a suitable treatment regimen for cancer and/or
(c) diagnosing cancer,
   comprising carrier means containing therein a set of primers for use in detecting the methylation status of a RecQ helicase family gene and preferably the WRN gene.

Any of the RecQ helicase family genes may be assessed using the kits of the invention. A more detailed discussion of family members is provided above.

This kit is preferably a kit for use in MSP and even more preferably a real-time detection version of MSP.

Thus, the kit includes suitable primers for determining whether the RecQ helicase family gene and preferably the WRN gene is methylated. These primers may comprise any of the primers discussed in detail in respect of the various methods of the invention which may be employed in order to determine the methylation status of the RecQ helicase family gene and preferably the WRN gene.

In one embodiment, the kit of the invention further comprises a reagent which modifies unmethylated cytosine. Such a reagent is useful for distinguishing methylated from unmethylated cytosine residues. In a preferred embodiment, the reagent comprises bisulfite, preferably sodium bisulfite. This reagent is capable of converting unmethylated cytosine residues to uracil whereas methylated cytosines remain unconverted. This difference in residue may be utilised to distinguish between methylated and unmethylated nucleic acid in a downstream process, such as PCR using primers which distinguish between cytosine and uracil (cytosine pairs with guanine, whereas uracil pairs with adenine).

In a real-time detection embodiment, the kit may further comprise probes for real-time detection of amplification products. These probes may simply be used to monitor progress of the amplification reaction in real-time and/or they may also have a role in determining the methylation status of the RecQ helicase family gene, in particular the WRN gene, themselves. Thus, the probes may be designed in much the same fashion as the primers to take advantage of sequence differences following treatment with a suitable reagent such as sodium bisulphite dependent upon the methylation status of the appropriate cytosine residues (found in CpG dinucleotides).

The probes may comprise any suitable probe type for real-time detection of amplification products. Non-limiting examples include use of TAQMAN probes and/or MOLECULAR BEACONS probes and/or AMPLIFLUOUR primers and/or LIGHT-CYCLER and/or FRET probes and/or SCORPION primers. All of these technologies are well characterised in the art and the design of suitable probes is routine for one of skill in the art. Notably, however, with the AMPLIFLUOUR and SCORPION embodiments, the probes are an integral part of the primers which are utilised. The probes are typically fluorescently labelled, although other label types may be utilised as appropriate.

In one embodiment, the primers in the kit comprise, consist essentially of, or consist of primers which are capable of amplifying methylated and/or unmethylated DNA following bisulfite treatment which DNA comprises, consists essentially of, or consists of the nucleotide sequence set forth as SEQ ID NO: 1.

In a specific embodiment, the primers in the kit comprise, consist essentially of, or consist of primers comprising, consisting essentially of, or consisting of the following nucleotide sequences for the purposes of amplifying methylated DNA (following bisulphite treatment):

| | |
|---|---|
| SEQ ID NO: 4 | 5′-CGG GTA GGG GTA TCG TTC GC-3′(sense) |
| and/or | |
| SEQ ID NO: 5 | 5′-AAC GAA ATC CAC CGC CCG CC-3′(antisense). |

In a specific embodiment, the primers in the kit comprise, consist essentially of, or consist of primers comprising, consisting essentially of, or consisting of the following nucleotide sequences for the purposes of amplifying unmethylated DNA (following bisulphite treatment):

| | | |
|---|---|---|
| SEQ ID NO: 2 | 5′-GTA GTT GGG TAG GGG TAT TGT TTG T-3′ | (sense) |
| and/or | | |
| SEQ ID NO: 3 | 5′-AAA CAA AAT CCA CCA CCC ACC CC-3′ | (antisense) |

In a further embodiment, in which bisulphite sequencing is utilised in order to determine the methylation status of the RecQ helicase family gene and preferably the WRN gene, the kit comprises primers for use in sequencing through the important CpG islands in the RecQ helicase family gene, and preferably the WRN gene. Thus, primers may be designed in both the sense and antisense orientation to direct sequencing across the promoter region of the gene.

In one embodiment, the primers in the kit comprise, consist essentially of, or consist of primers which are capable of sequencing of DNA following bisulfite treatment which DNA comprises, consists essentially of, or consists of the nucleotide sequence set forth as SEQ ID NO: 1.

In one embodiment, bisulphite sequencing may be carried out by using sequencing primers which comprise, consist essentially of or consist of the following sequences, and which may be used in isolation or in combination to sequence both strands:

| | | |
|---|---|---|
| SEQ ID NO: 6 | 5′-AGG TTT TTA GTY GGY GGG TAT TTA-3′ | (sense) |
| and/or | | |
| SEQ ID NO: 7 | 5′-AAC CCC CTC TTC CCC TCA-3′ | (antisense) |

As discussed with respect to the methods of the invention, suitable controls may be utilised in order to act as quality control for the methods. Accordingly, in one embodiment, the kit of the invention further comprises, consists essentially of or consists of one or more control nucleic acid molecules of which the methylation status is known. These (one or more) control nucleic acid molecules may include both nucleic acids which are known to be, or treated so as to be, methylated and/or nucleic acid molecules which are known to be, or treated so as to be, unmethylated. One example of a suitable internal reference gene, which is generally unmethylated, but may be treated so as to be methylated, is β-actin.

Furthermore, the kit of the invention may further comprise, consist essentially of or consist of primers for the amplification of the control nucleic acid. These primers may be the same primers as those utilised to monitor methylation in the test sample in a preferred embodiment. Thus, the control nucleic acid may comprise a RecQ helicase family gene, in particular the WRN gene, for example taken from normal tissues in which it is known to be unmethylated. The control nucleic acid may additionally comprise a RecQ helicase family gene, in particular the WRN gene in methylated form, for example as methylated by a methyltransferase enzyme such as SssI methyltransferase for example.

Suitable probes for use in determining the methylation status of the control nucleic acid molecules may also be incorporated into the kits of the invention. The probes may comprise any suitable probe type for real-time detection of amplification products. Non-limiting examples include use of TAQMAN probes and/or MOLECULAR BEACONS probes and/or AMPLIFLUOUR primers and/or LIGHT-CYCLER and/or FRET probes and/or SCORPION primers. All of these technologies are well characterised in the art and the design of suitable probes is routine for one of skill in the art. Notably, however, with the AMPLIFLUOUR and SCORPION embodiments, the probes are an integral part of the primers which are utilised.

The kits of the invention may additionally include suitable buffers and other reagents for carrying out the claimed methods of the invention. Thus, the discussion provided in respect of the methods of the invention as to the requirements for determination of the methylation status of a RecQ helicase family gene, and in particular the WRN gene, apply mutatis mutandis here.

In one embodiment, the kit of the invention further comprises, consists essentially of, or consists of nucleic acid amplification buffers.

The kit may also additionally comprise, consist essentially of or consist of enzymes to catalyze nucleic acid amplification. Thus, the kit may also additionally comprise, consist essentially of or consist of a suitable polymerase for nucleic acid amplification. Examples include those from both family A and family B type polymerises, such as Taq, Pfu, Vent etc.

The various components of the kit may be packaged separately in separate compartments or may, for example be stored together where appropriate.

The kit may also incorporate suitable instructions for use, which may be printed on a separate sheet or incorporated into the kit packaging for example.

The invention will now be described with respect to the following non-limiting examples.

### DESCRIPTION OF THE DRAWINGS

FIG. 1. Analysis of WRN CpG island promoter methylation status and gene function in human cancer cell lines.

A, Upper: Schematic depiction of the WRN CpG island around the transcription start site (long black arrow). CpG dinucleotides are represented as short vertical lines. Location of bisulfite genomic sequencing PCR primers are indicated as white arrows and methylation-specific PCR primers as grey arrows.

Lower: Results of bisulfite genomic sequencing of 12 individual clones for different normal tissues and human cancer cell lines. Presence of a methylated cytosine is indicated by a black square and presence of an unmethylated cytosine by a white square.

B, Methylation-specific PCR carried out on the WRN gene in human cancer cell lines. The presence of a PCR band under the lane M indicates methylated WRN genes, whilst the presence of a PCR band under the lane U indicates unmethylated genes. In Vitro Methylated DNA (IVD) is used as positive control for methylated DNA.

C, Reverse transcription-PCR analysis of WRN expression. Treatment with the demethylating agent (ADC+lanes) reactivates WRN gene expression.

D, Western-blot analysis of WRN expression. The WRN hypermethylated cell lines HCT-116, MDA-MB-231 and U937 do not express the WRN protein or have minimal expression (MDA-MB-231). Treatment with the demethylating agent ADC reactivates WRN gene expression. WS−/− cells are shown as negative control.

E, Immunohistochemistry of WRN. The WRN methylated cell lines MDA-MB-231 and U937 and the mutant WS−/− cells do not show staining for the WRN protein, in comparison with the unmethylated K562 and MDA-MB-468 cells.

F, Immunofluorescence analysis of WRN expression. The methylated cell lines COLO-205 and U937 and the mutant WS−/− cells do not show any staining for the WRN protein, in comparison with the unmethylated MCF-7 cells. Treatment with the demethylating agent (ADC) restore protein expression. DAPI is shown as a marker of nuclear DNA.

G, Exonuclease activity assay in WRN-immunoprecipitated cell resolved on denaturing polyacrylamide gels. The 3′-recessed duplex substrate used for exonuclease studies was degraded more extensively in WRN unmethylated cells (MCF-7, HL60) than in WRN methylated (U937, MDA-MB-231) or mutated cells (WRN−/−).

FIG. 2. Tumor suppressor-like properties of WRN re-introduction.

A, Colony formation assay.

Upper: WRN expression monitored by RT-PCR in untransfected and WRN-transfected MDA-MB-231 cells monitored by RT-PCR.

Lower: Densitometric quantification of the colony formation density of MDA-MB-231 cells transfected with the empty vector or with WRN. Three independent experiments were carried out.

B, Example of the colony focus assay after a 2-week selection of G418 and staining with methylene blue.

C, D and E, Effect of WRN transfection on the in vivo growth of MDA-MB-231 cells. Tumor weight (FIG. 2C) and size (FIG. 2D) was monitored over time. Female athymic nude mice 8 days after injection of 106 MDA-MB-231 cells are shown in FIG. 2E. Note the large tumor on the left flank, corresponding to empty vector-MDA-MB-231 cells, and the small tumor on the opposite flank, corresponding to WRN-MDA-MB-231 cell injection. Tumors were excised cautiously to avoid skin contamination and then weighed. Tumor detail and weight in mg is shown.

FIG. 3. Hypermethylation-deficient WRN cancer cells are sensitive to inhibitors of topoisomerase I and DNA damaging agents.

A and B: Induction of apoptosis measured by flow cytometry in unmethylated (MCF-7), methylated (MDA-MB-231) and mutated (WS−/−) WRN cells at increasing concentrations of camptothecin (FIG. 3B) and mytomycin C (FIG. 3A). MDA-MB-231 and WS−/− cells are highly sensitive in comparison with MCF-7.

Restoration of WRN expression in MDA-MB-231 cells induces resistance to apoptosis by both drugs (see MDA-MB-231-WRN).

C: Chromosomal breakage measured by cytogenetic analysis of metaphase chromosomes. Untreated MDA-MB-231 cells have undetectable fragility (labelled MDA-MB-231), 50 mg/ml mytomycin C treated cells undergo a massive breakage (labelled empty vector), and MDA-MB-231 cells transfected with the WRN gene (labelled WRN) display resistance to the genome damage.

D: Quantification of chromosomal breakage induced by mytomycin C in cells proficient (HL60, MCF-7) or deficient in WRN function by mutation (WS−/−) or methylation (MDA-MB-231). Transfection of the WRN gene in MDA-MB-231 cells provokes resistance to the genomic damage induced by the drug (compare MDA-MB-231-MOCK with MDA-MB-231-WRN).

FIG. 4A. Analysis of WRN CpG island hypermethylation in primary human malignancies by methylation-specific PCR. The presence of a PCR band under the lane M indicates methylated genes, whilst the presence of a PCR band under the lane U indicates unmethylated genes. Normal lymphocytes (NL) and In Vitro Methylated DNA (IVD) are used as negative and positive control for unmethylated and methylated genes, respectively.

FIG. 4B: Kaplan-Meier analysis of WRN promoter hypermethylation in patients with colorectal cancer treated with irinotecan and its impact on survival. A significant increased overall survival is observed in patients with WRN methylation. Numbers of patients at each time are shown at the bottom of the figure.

FIG. 5: CpG island of the WRN gene. Transcription start site is shown in light grey shading and the potential sites of methylation are shown in dark grey shading.

FIG. 6: Induction of apoptosis measured by flow cytometry at increasing concentrations of camptothecin (FIG. 6B) and mitomycin C (FIG. 6A) in the leukaemia cell line U937 that has WRN hypermethylation. U937 untransfected cells or U937-mock transfected cells are highly sensitive, whilst restoration of WRN expression in U937 cells by re-introduction of the WRN gene induces resistance to apoptosis by both drugs.

### EXPERIMENTAL SECTION

### Introduction

Werner syndrome (WS) is an inherited disorder characterized by premature onset of aging, genomic instability and increased cancer incidence. The disease is caused by loss of function mutations of the WRN gene, a member of the RecQ helicase with both helicase and exonuclease activities. However, despite its putative tumor-suppressor function, little is known of the contribution of WRN to human sporadic malignancies. Here, we report that WRN function is abrogated in human cancer cells by transcriptional silencing associated with CpG island promoter hypermethylation. We also show that, at the biochemical and cellular levels, the epigenetic inactivation of WRN leads to the loss of WRN-associated exonuclease activity and increased chromosomal instability and apoptosis induced by inhibitors of topoisomerase. The described phenotype is reversed by the use of a DNA demethylating agent or by the re-introduction of WRN into cancer cells displaying methylation-dependent silencing of WRN. Furthermore, the restoration of WRN expression induces tumor-suppressor-like features, such as reduced colony formation density and inhibition of tumor growth in nude mouse xenograft models. Screening a large collection of human primary tumors (n=630) from different cell types, we found that WRN CpG island hypermethylation was a common event in epithelial and mesenchymal tumorogenesis. Most important, WRN hypermethylation in colorectal tumors was a strong predictor of good clinical response to the camptothecin analogue irinotecan, a topoisomerase inhibitor commonly used in the clinical setting for the treatment of this tumor type. These findings highlight the importance of WRN epigenetic inactivation in human cancer, leading to enhanced chromosomal instability and hypersensitivity to chemotherapeutic drugs.

Here we have demonstrated for the first time that WRN undergoes CpG island promoter methylation-associated gene silencing in human cancer cells. The hypermethylation of the WRN promoter leads to its loss of expression and hypersensitivity to topoisomerase inhibitors and DNA damaging agents. The epigenetic loss of WRN function can be rescued by the use of DNA demethylating agents. Furthermore, the reintroduction of WRN into those transformed cell lines with WRN-deficiency due to hypermethylation provokes a reduction in colony formation and a decrease in growth of tumor xenografts, supporting the hypothesis of a tumor-suppressor role for WRN. The analysis of a large panel of human primary tumors (n=630) shows that WRN CpG island hypermethylation is a common event in tumorigenesis. Most important, for colorectal cancer, the presence of aberrant methylation at the WRN promoter predicts improved survival in those patients treated with irinotecan, a topoisomerase inhibitor commonly used in this neoplasm. These findings underline the significance of WRN as a caretaker of our genome with tumor suppressor activity, and identify epigenetic silencing of WRN as one key step in cancer development that may have an important clinical impact for the treatment of these patients.

### Methods

### Cell Lines and Tumor Samples.

The eleven human cancer cell lines examined in this study were obtained from the American Type Culture Collection (ATCC) (Rockland, Md., USA) and the German Collection of Microorganisms and Cell Cultures (DSMZ, Braunschweig, Germany). The cell lines represented three different types of malignancies, colon (HCT-116, COLO-205), breast (MCF-7, MDA-MB-468, MDAMB-231) and leukaemia (HL-60, KG1a, U937, REH, Jurkat, K562). Cell lines were maintained in appropriate media and treated with 1 μM 5-aza-2′-deoxycytidine (Sigma) for 3 days to achieve demethylation, as previously described (Herman et al., 1998; Ropero et al., 2004). WS−/− cells (AG11395) were obtained from the Coriel Cell Repositories (Camden, N.J., USA). The collection of primary tumor samples analyzed has been described elsewhere (Esteller et al., 2001; Ropero et al., 2004).

### DNA Methylation Analysis of the WRN Gene.

We established WRN CpG island methylation status by PCR analysis of bisulfite-modified genomic DNA, which induces chemical conversion of unmethylated, but not methylated, cytosine to uracil, using two procedures. First, methylation status was analyzed by bisulfite genomic sequencing of both strands of the corresponding CpG islands. The primers used for WRN were 5′-AGG TTT TTA GTY GGY GGG TAT TTA-3′ (sense) and 5′-AAC CCC CTC TTC CCC TCA-3′ (antisense), located −209 bp and +164 bp from the transcription start site.

The second analysis used methylation-specific PCR using primers specific for either the methylated or modified unmethylated DNA. Primer sequences of WRN for the unmethylated reaction were 5′-GTA GTT GGG TAG GGG TAT TGT TTG T-3′ (sense) (SEQ ID. NO: 2) and 5′-AAA CAA AAT CCA CCA CCC ACC CC-3′ (antisense) (SEQ ID. NO. 3) and for the methylated reaction, 5′-CGG GTA GGG GTA TCG TTC GC-3′ (sense) (SEQ ID. NO. 4) and 5′-AAC GAA ATC CAC CGC CCG CC-3′ (antisense) (SEQ ID. NO. 5). Primers were located −36 (sense) and +129 (antisense) from the transcription start site. The annealing temperature for both unmethylated and methylated reactions was 62° C. DNA from normal lymphocytes treated in vitro with SssI methyltransferase was used as a positive control for methylated alleles. DNA from normal lymphocytes was used as a positive control for unmethylated alleles. PCR products were loaded onto non-denaturing 3% polyacrylamide gels, stained with ethidium bromide and visualized under a UV transilluminator.

### WRN RNA and Protein Analysis by Western Blotting, Immunohistochemistry and Immunofluorescence.

RNA was isolated using TRIzol (Life Technologies, Gaithersburg, Md., USA). 2 μg of RNA was reverse-transcribed using SuperScript II reverse transcriptase (Gibco/BRL) and amplified using specific primers for WRN (forward: 5′-GCA TGT GTT CGG AAG AGT GTT T-3-(SEQ ID. NO: 6), reverse: 5′-TGA CAT GGA AGA AAC GTG GAA-3 (SEQ ID. NO: 7)) and for GAPDH. PCR was performed for 25 cycles (94° C. for 30 s, 57° C. for 30 and 72° C. for 30 s) in a final volume of 25 μl containing 1×PCR buffer (Gibco/BRL), 1.5 mM MgCl2, 0.3 mM of dNTP, 0.25 mM of each primer and 2 U of taq polymerase (Gibco/BRL). RT-PCR primers were designed between different exons and encompassing large introns to avoid any amplification of genomic DNA. Cell lysates for protein analysis were prepared and analyzed by western blotting using the WRN antibody ab200 (Rabbit Polyclonal, Abcam). Equal loading was tested by reprobing with a polyclonal antibody against human b-actin. Gels were cast using the XCell SureLock Mini-Cell system (Invitrogen Corp./NOVEX, Carlsbad, Calif.) and developed using ECL immunodetection reagents (Amersham Pharmacia Biotech, Piscataway, N.J.). For immunohistochemistry, antigen retrieval was achieved by heat treatment in a pressure-cooker for 2 minutes in 10 mmol/L citrate buffer (pH 6.5) (Agrelo et al., 2005). After incubation with the described WRN antibody, immunodetection was performed with EnVision-HRP (DakoCytomation, Copenhagen, Denmark) and peroxidase activity was developed using 3,3-diaminobenzydine chromogen as substrate. Sections were counterstained with hematoxylin. For immunofluorescence, cells were grown on coverslips in P60 dishes, fixed in 4% formaldehyde and stained as previously described (Ropero et al., 2004; Agrelo et al., 2005).

### Exonuclease Assay

The exonuclease enzymatic activity was measured as previously described (Brosh et al., 2001). Exonuclease assay reaction mixtures (10 μl) contained 40 mM Tris (pH 7.4), 5 mM MgCl2, 1 mM dithiothreitol, 0.1 mg/ml BSA, 1 mM ATP, and lysates from 8,000 cells immunoprecipitated with WRN antibody H-300 (Rabitt IgG, Santacruz)) and prepared in ice-cold 50 mM Tris-HCl, pH 7.4, 150 mM NaCl, 11 Nonidet P-40, and 5 mM EDTA containing protease inhibitors. The DNA exonuclease substrate consisted of a double-stranded DNA molecule with one blunt end and one recessed 3′ end (5′ overhang of 20 nucleotides) and its amount in the reaction mixture was ˜3 fmol. Reactions were incubated at 37° C. for 60 min. Reactions were stopped by the addition of an equal volume of formamide loading buffer (80% formamide, 0.5×Tris-borate EDTA, 0.1% bromphenol blue, and 0.1% xylene cyanol). The digestion products of these reactions were separated on 15% denaturing polyacrylamide gels, visualized using a PhosphorImager (Molecular Dynamics), and quantitated using ImageQuant software (Molecular Dynamics).

### Flow Cytometry and Cytogenetic Assays.

The percentage of apoptotic cells was determined by flow cytometry using Vybrant® Apoptosis Assay Kit #4-YO-PRO®-1/propidium iodide (Molecular Probes/Invitrogen). Briefly, cells were washed twice with ice cold PBS and resuspended in PBS containing YO-PRO®-1/propidium iodide. Apoptotic cells were identified by flow cytometry after 20 min of incubation. Cytogenetic assay was performed as previously described (Herranz et al., 2006) analyzing one-hundred metaphases for each experimental condition.

### WRN Transfection

The WRN coding sequence corresponding to the cDNA from a BLCL lymphoblastoid-EBV immortalized cell line was amplified by PCR and directly cloned into the pGEM-T Easy Vector (Promega, Winsconsin USA). The WRN insert was subcloned into the pEGFP-N1 expression vector (Invitrogen) and confirmed by sequencing. For transfection experiments we used the pEGFP-N1 vector containing the WRN gene, or the pEGFP-N1 empty vector. Transfection of MDA-MB-231 and U937 cells was performed by electroporating 107 cells in 0.8 ml PBS with 40 μg of the vector at 250 V and 975 μF. After electroporation, cells were washed with PBS and seeded with 106 cells/ml in fresh medium containing 20% FBS. Transfected cells were selected by the addition of G418 (600 μg/ml).

### Colony Formation Assay.

Colony formation on methylcellulose medium (Stemcell Technologies) was assayed. Transfected cells were added to a medium containing 80% methylcellulose and 20% conditioned medium from MDA-MB-231 cultures and 600 μg/ml of G418. The mixture was then placed in a six-well plate and incubated for 15 days. Colonies containing more than 20 cells were scored as positive.

### Mouse Xenograft Model.

Six-week-old female athymic nude mice nu/nu (Harlam Sprague Dawley, Indianapolis, Ind.), housed under specific pathogen-free conditions (Institutional Animal Welfare Committee Agreement), were used for MDA-MB-231 tumor xenografts. Ten specimens were used. Both flanks of each animal were injected with 107 cells in a total volume of 200 μl of PBS. The right flank was always used for WRN-MDA-MB-231-transfected cells and the left for empty-vector MDA-MB-231 control cells. Tumor development at the site of injection was measured daily. Animals were sacrificed at 30 days. The tumors were then excised and weighed.

### Statistical Analysis.

Contingency tables were analyzed by Fisher's exact test. Overall survival curves were estimated by the Kaplan-Meier method and were compared with the use of the log-rank test. All statistical analyses were performed using the SPSS version 10.1 program (SPSS Inc, Chicago, Ill.).

### Results

### WRN Promoter CpG Island Hypermethylation Leads to Gene Inactivation.

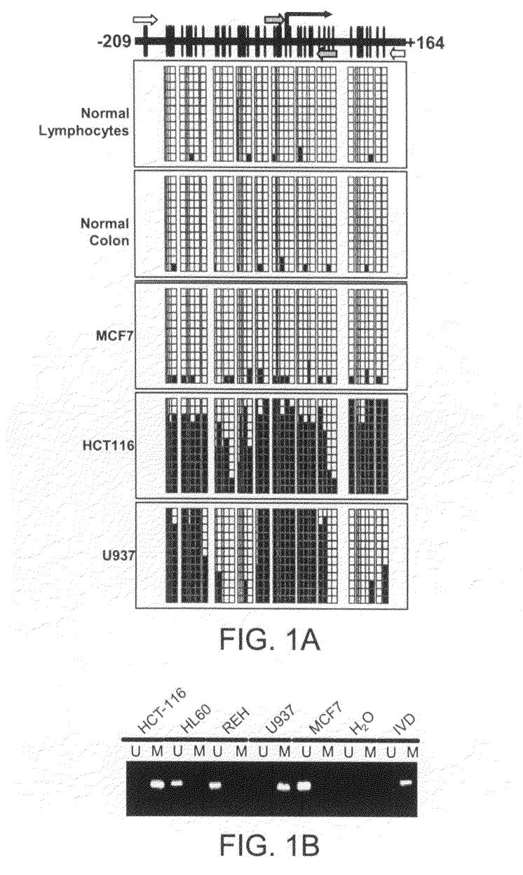

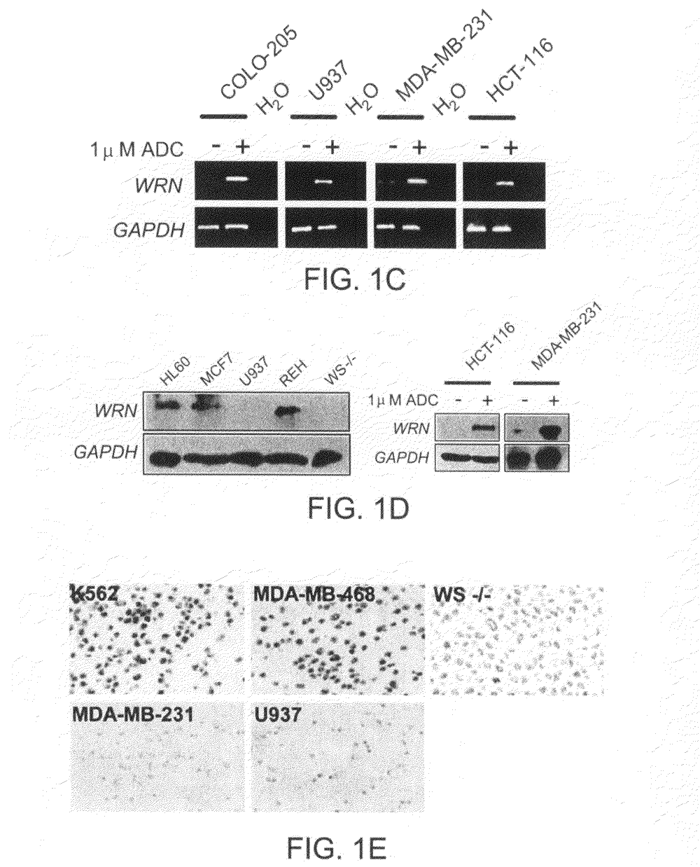

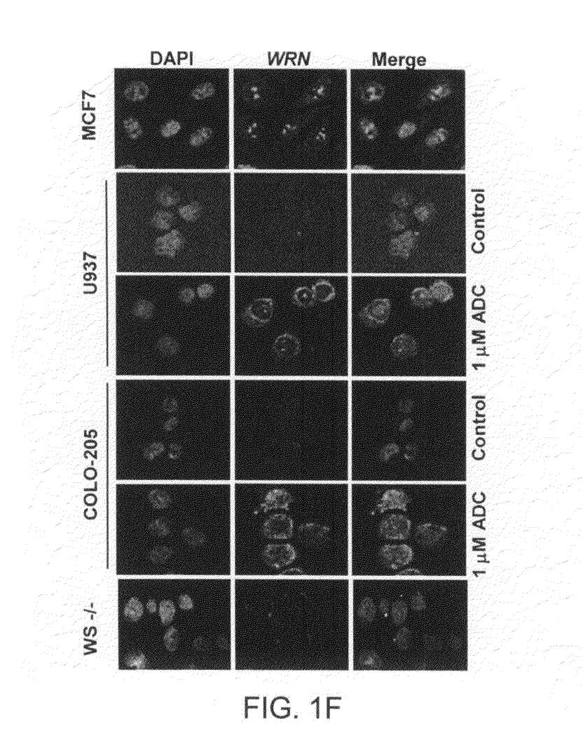

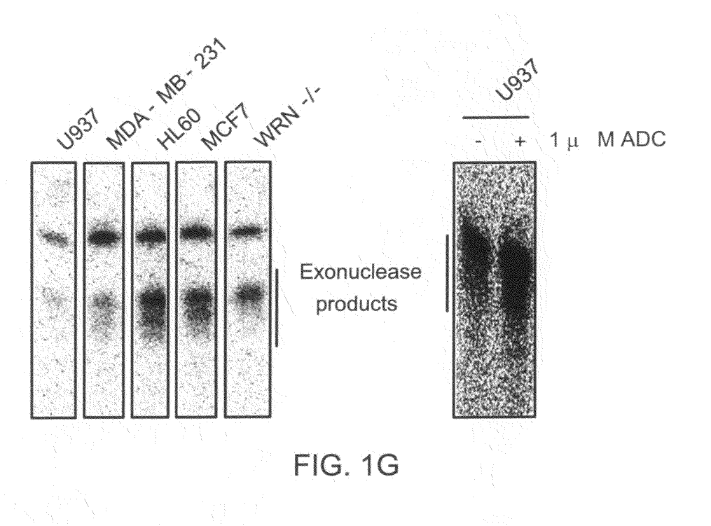

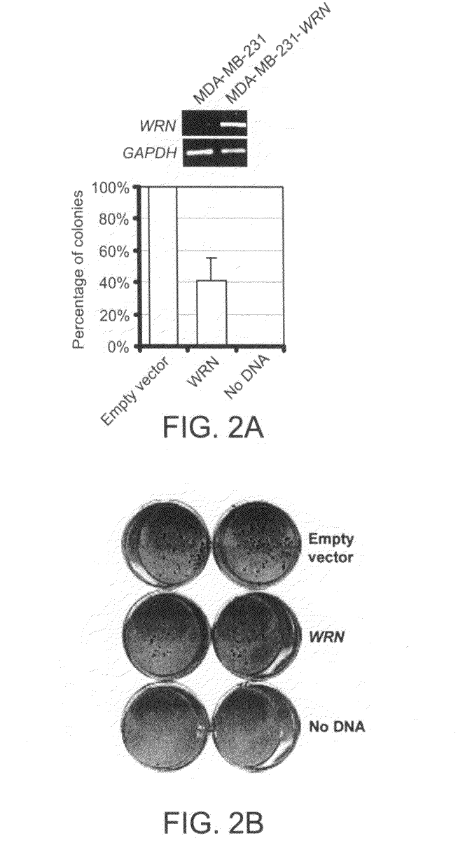

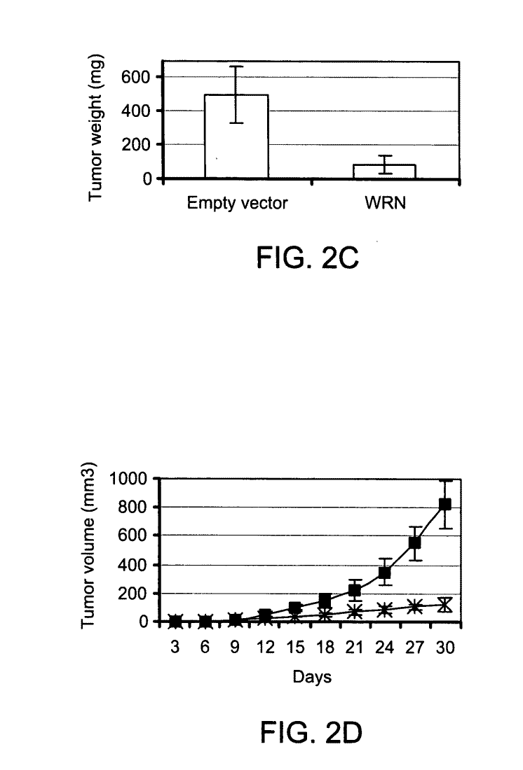

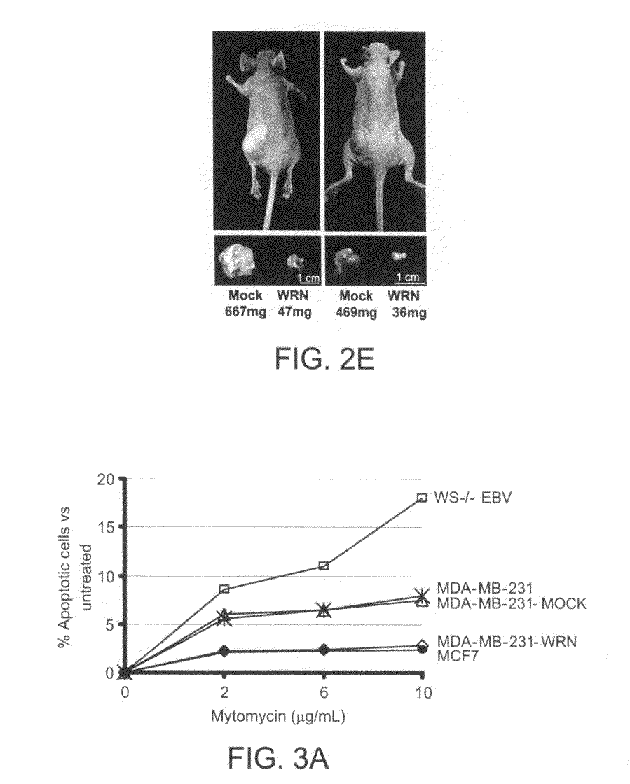

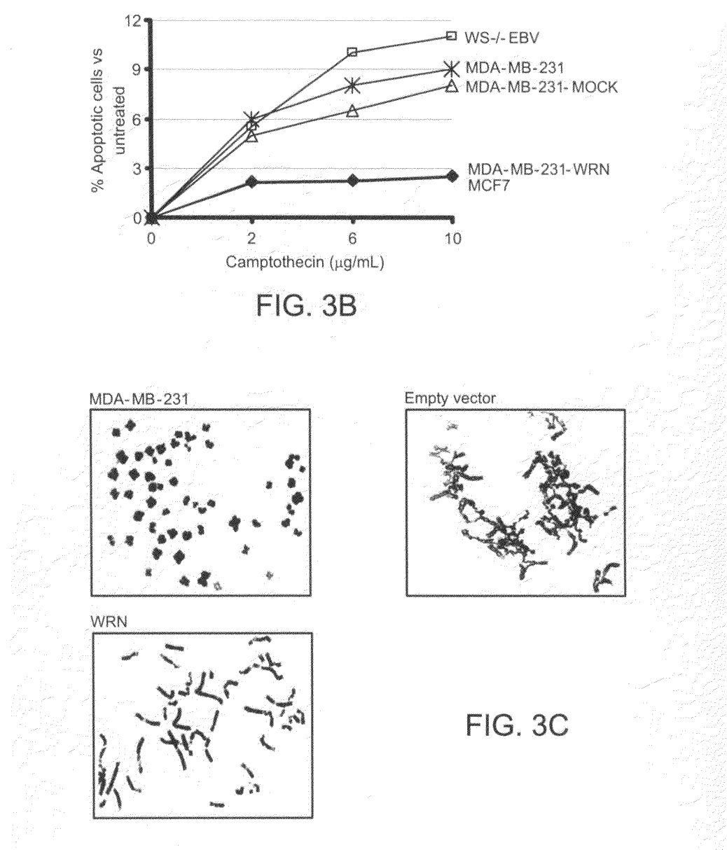

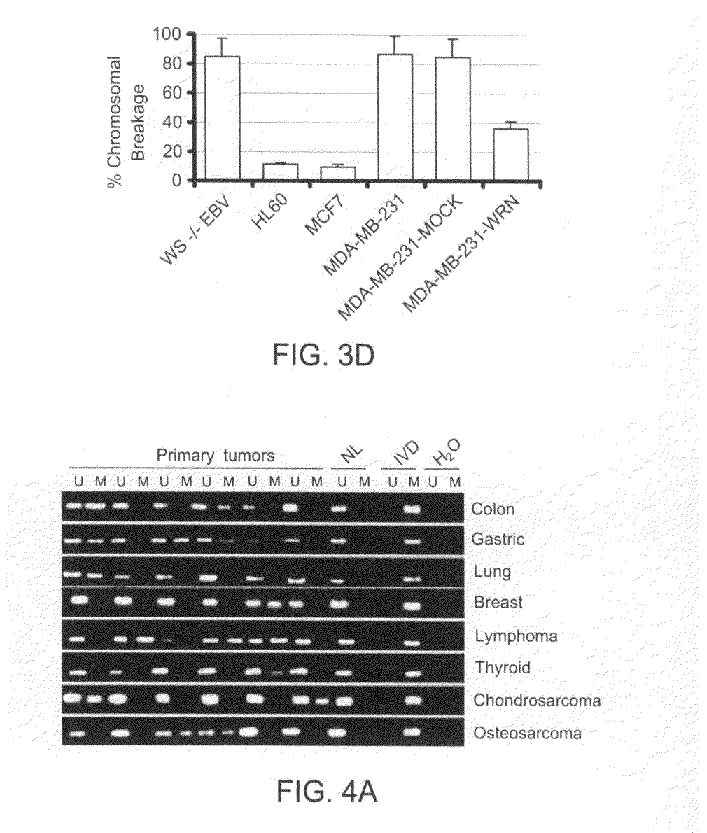

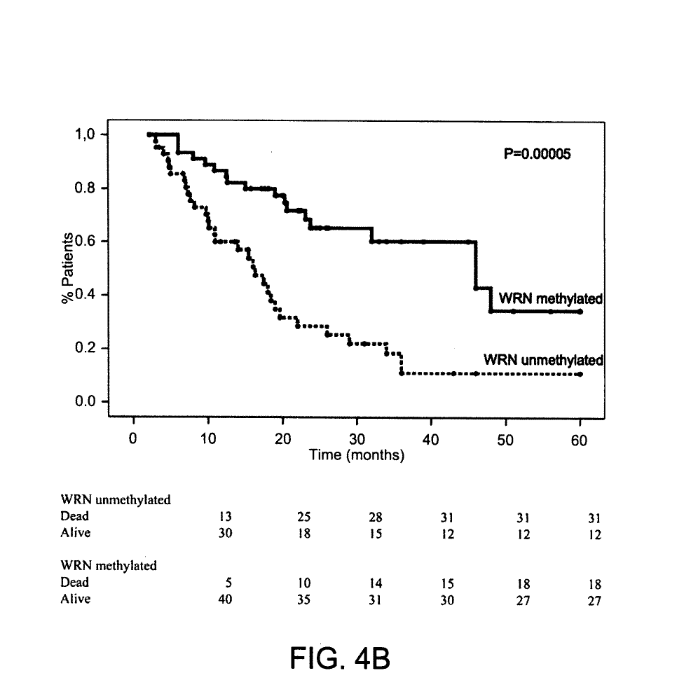

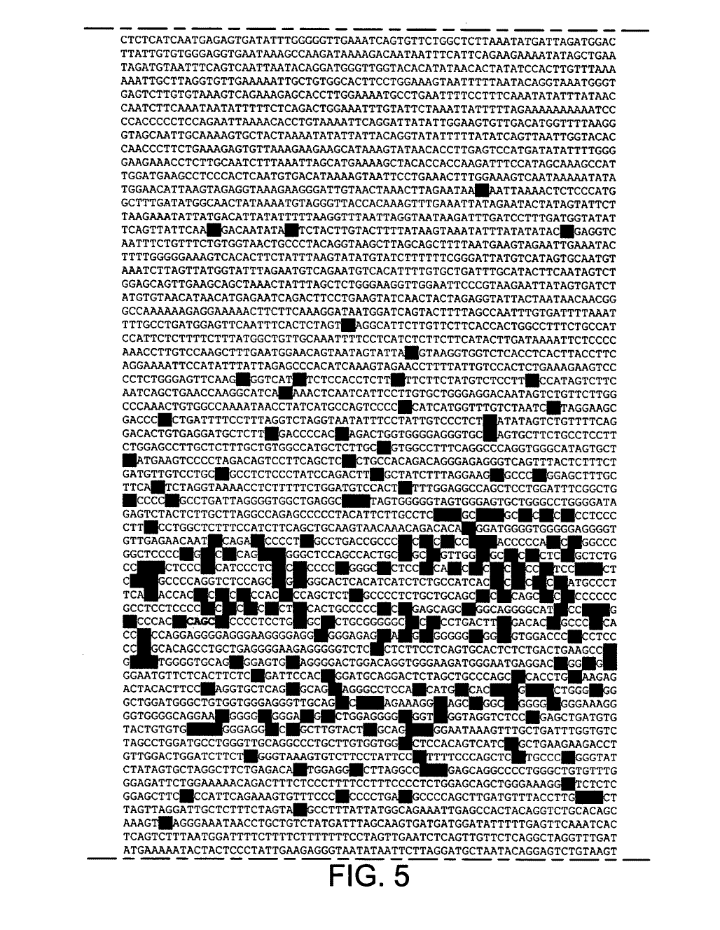

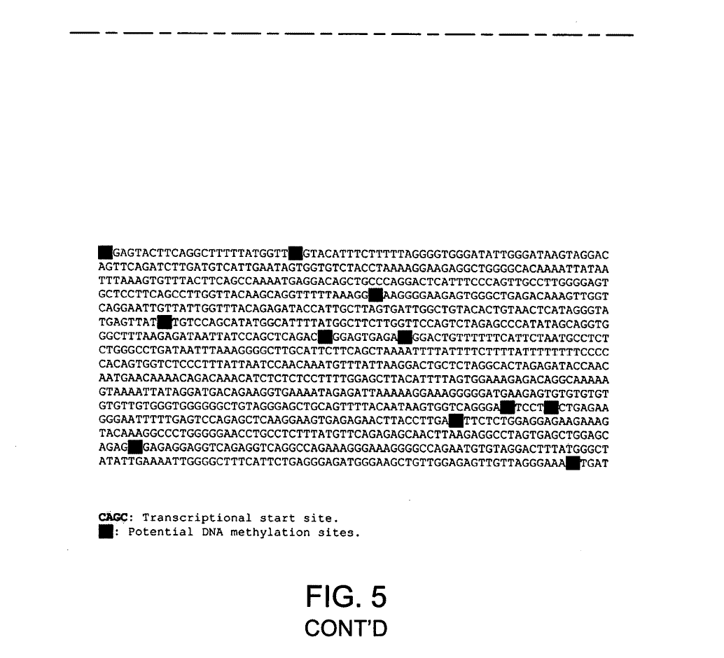

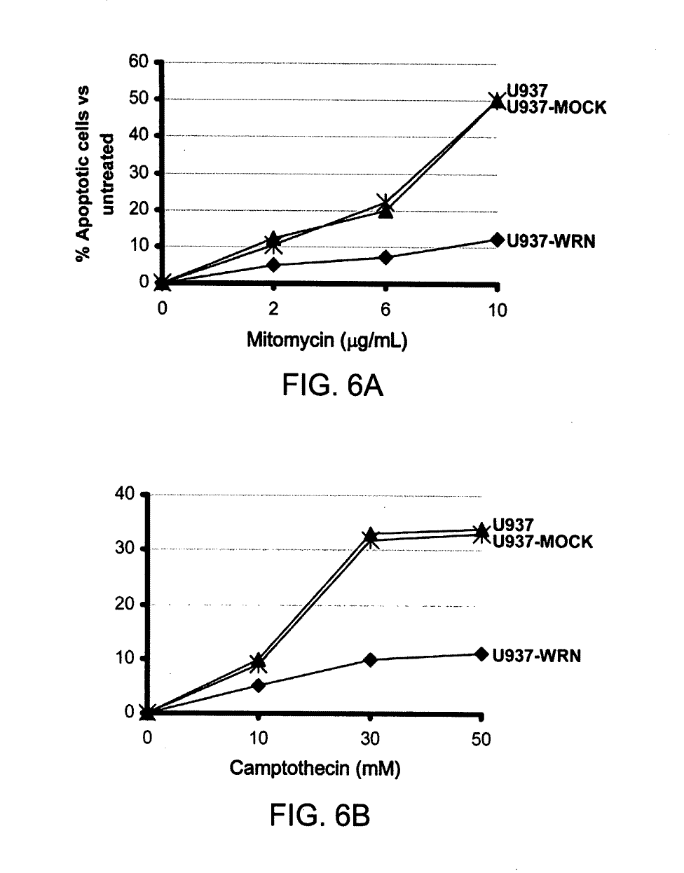

WRN is a gene candidate for hypermethylation-associated inactivation in human cancer since a 5′-CpG island is located around the transcription start site (FIG. 1A).

To analyze the methylation status of the promoter-associated CpG island, we screened eleven human cancer cell lines from three different cell types of malignancies, colon (HCT-116, COLO-205), breast (MCF-7, MDA-MB-468, MDA-MB-231) and leukaemia (HL-60, KG1a, U937, REH, Jurkat, K562) using bisulfite genomic sequencing and methylation-specific PCR targeted to the area surrounding the transcription start site, as described in Methods. WRN CpG island promoter hypermethylation was found in four cancer cell lines: HCT-116, COLO-205, MDA-MB-231 and U937 (FIGS. 1A and 1B). All normal tissues analyzed, including lymphocytes, bone marrow, breast, colon and skin, were completely unmethylated at the WRN promoter (FIGS. 1A and 1B).

Having noted WRN promoter hypermethylation in cancer cell lines, we assessed the association between this epigenetic aberration and the putative transcriptional inactivation of the WRN gene at the RNA and protein levels. The cancer cell lines, HCT-116, COLO-205, MDA-MB-231 and U937 hypermethylated at the WRN CpG island, did not express (HCT-116, COLO-205 and U937) or had minimal expression (MDA-MB-231) of the WRN RNA transcript, as determined by reverse-transcription PCR(RT-PCR) (FIG. 1C) and WRN protein, as determined by western-blot (FIG. 1D), immunohistochemistry (FIG. 1E) and immunofluorescence (FIG. 1F). In contrast, MCF-7, MDA-MB-468, HL-60, K562, KG1a, Jurkat and REH, unmethylated at the WRN promoter, expressed WRN protein (FIGS. 1D, 1E and 1F).

We established a further link between WRN CpG island hypermethylation and its gene silencing by the treatment of the methylated cell lines with a DNA demethylating agent. The treatment of the HCT-116, COLO-205, MDA-MB-231 and U937 cell lines with the demethylating drug 5-aza-2′-deoxycytidine restored the expression of WRN RNA transcript and protein (FIGS. 1C, 1D and 1F). It is not only a matter of restoring gene expression, but also of rescuing gene functionality. This is exemplified by several genes that undergo methylation-associated silencing, such as the DNA repair gene hMLH1, the MDM2-regulator p14ARF, or the glycosyltransferase EXT-1, where treatment with the demethylating agent induced recovery of gene functions: DNA mismatch repair activity, sequestration of MDM2 and heparan sulfate biosynthesis respectively (Herman et al., 1998; Esteller et al., 2001; Ropero et al., 2004). As WRN is the only RecQ member that exhibits exonuclease activity (Huang et al., 1998), we examined the impact of WRN methylation-mediated silencing in this enzymatic function of WRN and the effect of restoring WRN expression by pharmacological means.

We observed a loss of exonuclease enzymatic activity in WRN-immunoprecipitated cell extracts of U937 and MDA-MB-231, all of them hypermethylated at the WRN promoter, compared with HL60 and MCF-7 cells, these last two having an unmethylated WRN promoter (FIG. 1G). Most important, treatment of U937 cells with the demethylating agent 5-aza-2′-deoxycytidine induced a significant increase in the WRN-associated exonuclease activity in these cells (FIG. 1G).

### Re-Introduction of WRN in Hypermethylation-Deficient Cancer Cell Lines has Tumor-Suppressor-Like Properties.

Although the tumor-suppressor gene features of WRN have been proposed before, we assayed the ability of WRN to function as a suppressor of tumor growth in our model, using the breast cancer cell line MDA-MB-231 with WRN methylation associated silencing. We first tested the inhibitory abilities of WRN in a colony-focus assay using G418 selection after transfection with the WRN gene (pEGFP-N1-WRN) or the empty vector (pEGFP-N1). WRN expression was monitored by RT-PCR (FIG. 2A, upper). Inhibition of tumor-cell growth was assessed by seeding MDA-MB-231-transfected cells on methylcellulose, incubating for 15 days, and then scoring the number of colonies formed (FIG. 2B). WRN re-expression demonstrated tumor-suppressing activity with a marked reduction of 59%+10 in colony-formation density with respect to the empty vector (FIG. 2A, lower).

We next tested the ability of WRN-transfected MDA-MB-231 cells to form tumors in nude mice compared with empty vector-transfected MDA-MB-231 cells. The same mice were subcutaneously injected with 106 WRN (right flank) or empty vector transfected (left flank) MDA-MB-231 cells (FIG. 2E). All mice were killed 30 days after the injection and the tumors were dissected and weighed. Cells transfected with the empty vector (MDA-MB-231/pEGFP-N1) formed tumors rapidly, but cells infected with the WRN expression vector (MDA-MB-231/pEGFP-N1-WRN) had much lower tumorigenicity (FIGS. 2C, D and E). At the time of sacrifice, tumors were six times larger in those mice with the empty vector, 500+167.3 milligrams, than in those xenografts arising in the mice transfected with WRN, 86+50.4 milligrams (FIG. 2C).

### Hypermethylation-Deficient WRN Cancer Cells are Sensitive to Inhibitors of Topoisomerase I and DNA Damaging Agents.

It is already well characterized that lymphoblastoid cells and fibroblasts established from WS patients (Okada et al., 1998; Pichierri et al., 2000; Lebel, 2001; Opresko et al., 2003; Lowe et al., 2004) and embryonic stem cell from WRN-deficient mice (Lebel and Leder, 1998) are hypersensitive to chromosomal-damage and apoptosis upon their exposure to topoisomerase inhibitors and DNA cross-linking drugs. It would be extremely interesting to know for clinical translational purposes if cancer cells with WRN-methylation associated silencing also display these functional features. To address this issue, we treated WRN-unmethylated and hypermethylated cancer cell lines with camptothecin (a topoisomerase I inhibitor) or mitomycin C (an interstrand crosslinker) and measured the apoptotic rate by flow cytometry and the chromosomal breakage by cytogenetic analysis.

For the apoptosis study, we observed that both camptothecin and mitomycin C were optimal inductors of apoptosis in the breast cancer cell line MDA-MB-231 and the leukaemia cell line U937 (FIG. 3A, 3B and FIG. 6), both of them having WRN promoter hypermethylation. The same phenomena was observed for the lymphoblastoid cell line from a WS patient used as a positive control (FIGS. 3A and 6). In a significant contrast, the breast cancer cell line MCF-7, unmethylated at the WRN promoter, was markedly resistant to camptothecin and mitomycin C-induced apoptosis (FIG. 3A). Most important, when we used WRN-transfected U937 and MDA-MB-231 cells, these resulting cells were now resistant to the apoptosis mediated by both drugs (FIGS. 3A, 3B and 6).

The results from the cytogenetic chromosomal breakage analysis mimic those obtained from the apoptotic assays. Human cancer cell lines with an unmethylated WRN promoter, such as the breast cancer MCF-7 or the leukemic HL60 cells demonstrated a minimal chromosomal breakage upon exposition to camptothecin (FIGS. 3C and D). In contrast, both the lymphoblastoid cells from a WS patient and the breast MDA-MB-231 cells with WRN aberrant methylation were extremely sensitive to the drug and a high number of chromosomal breakages, with even the characteristic formation of quatriradials chromosomes, were observed in metaphases (FIGS. 3C and D). Furthermore, when WRN-transfected MDA-MB-231 cells were exposed to camptothecin, these cells now acquired a resistance to experiment chromosomal breakage (FIGS. 3C and D).

### Profile of WRN CpG Island Hypermethylation in Human Primary Malignancies

Following the demonstration of the epigenetic loss of function of WRN in cancer-cell lines, we assessed the prevalence of WRN CpG island promoter hypermethylation in cancer patients. We examined 630 primary tumors corresponding to eleven different tissue types. WRN CpG island promoter hypermethylation was observed with different frequency among each class of neoplasm and was present both in epithelial and mesenchymal tumor. The highest prevalence of WRN hypermethylation for the epithelial tumors was found in colorectal cancer (37.9%, 69/182), followed by non-small cell lung (37.5%, 21/56), gastric (25%, 10/38), prostate (20%, 4/20), breast (17.2%, 10/58) and thyroid (12.5%, 4/32) tumors. Among haematological malignancies, promoter methylation of the WRN gene was often found in non-Hodgkin lymphoma (23.7%, 28/118), but was much less common in acute lymphoblastic leukaemia (9.5%, 2/21) and acute myeloblastic leukaemia (4.7%, 3/63). For the mesenchymal tumor types, WRN hypermethylation was present in chondrosarcomas (33.3%, 5/15) and osteosarcomas (11.1%, 3/27). Table 1 summarizes the data obtained from primary tumors and examples of the methylation analyses are illustrated in FIG. 4A.

TABLE 1
Profile of WRN CpG island hypermethyl-
ation in human primary malignancies
Primary tumours (n = 630)
a) Epithelial tumours

| Colon | NSCLC | Stomach | Prostate | Breast | Thyroid |
|---|---|---|---|---|---|
| 37.9% | 37.5% | 25% | 20% | 17.2% | 12.5% |
| 69/182 | 21/56 | 10/38 | 4/20 | 10/58 | 4/32 |

b) Haematological malignancies

| NHL | ALL | AML |
|---|---|---|
| 23.7% | 9.5% | 4.7% |
| 28/118 | 2/21 | 3/63 |

c) Mesenchymal tumours

| Chondrosarcomo | Osteosarcoma |
|---|---|
| 33.3% | 11.1% |
| 5/15 | 3/27 |

NSCLC = Non Small Cell Lung Cancer
NHL = Non Hogkins Lymphoma
ALL = Acute Lymphoblastic Leukaemia
AML = Acute Myeloid Leukaemia

### WRN Hypermethylation in Colon Cancer Patients Predicts Good Response to the Camptothecin Analogue Irinotecan

Since cell lines from WS patients are extremely sensitive to the drug camptothecin (Okada et al., 1998; Pichierri et al., 2000; Lebel, 2001; Opresko et al., 2003; Lowe et al., 2004) and we have also described above that human cancer cells with WRN-methylation associated silencing are also very sensitive to the same drug, we wondered about the possibility to translate these observations to clinical samples. In this regard, one camptothecin analogue, irinotecan (CPT-11), has been approved for clinical use in the treatment of colon cancer, a tumor type where we found a significant rate of WRN CpG island hypermethylation.

Thus, we assessed if the presence of WRN promoter hypermethylation was a predictive marker of response to irinotecan in colorectal cancer patients treated with this drug. We selected a similar number of WRN-hypermethylated (n=45) and unmethylated (n=43) primary colorectal tumors from patients treated with irinotecan where a long clinical follow-up was available. We found that the median time to the death of the patient was 39.4 months for WRN methylated colon tumors and just 20.7 months for WRN unmethylated colon tumors.

Thus, the presence of WRN CpG island promoter hypermethylation was a significant strong predictor of increased overall survival in colon cancer patients treated with irinotecan (Kaplan-Meier p=0.00005; 95% confidence interval 25.4-35.2). The data is shown in FIG. 4B.

### Discussion

Aging is the main risk factor associated with cancer development (Ershler and Longo, 1997). Thus, it makes sense that the inactivation of a gene involved in “preventing” the aging process occurs in cancer cells. We have recently found a first example of this paradigm: lamin A/C (LMNA) is mutated in atypical WS, where the WRN gene is wildtype (Chen et al., 2003), and the LMNA gene undergoes methylation associated silencing in heamotological neoplasms (Agrelo et al., 2005). This is now also the case for the WRN gene.

Patients with WS display a remarkable number of clinical signs and symptoms associated with premature aging, including graying of the hair, cataracts, osteoporosis diabetes and atherosclerosis starting as early as the second or third decade of life (Epstein et al., 1966; Salk, 1982). Most important, WS patients display a high incidence of malignant neoplasms (Epstein et al., 1966; Salk, 1982). But what makes the case even more interesting it is that the tumor type of neoplasms appearing in WS patients is remarkably different that the one observed outside the syndrome: the ratio of mesenchymal vs epithelial cancers is 1:1, as compared with 1:10 in the normal ageing population (Chen and Oshima 2002; Hickson et al., 2003). Thus, it seems that the accelerated ageing process in WS patients contributes per se in part to the higher incidence of tumors, but the specific loss of the WRN gene really confers a particular tumor-type prone phenotype, in a similar fashion that is observed by other familial tumor suppressor genes with DNA repair function, such as hMLH1 or BRCA1 (Nagy et al., 2004). In this scenario, our discovery that the WRN gene undergoes epigenetic inactivation by CpG island promoter hypermethylation across different tumor types of both mesenquimal and epithelial origin may provide another clue to the contribution of the WRN protein to the tumorigenic process.

Different new avenues of research now may be open in the understanding of the relevance of the WRN gene in cancer development and progression. We have demonstrated that the presence of hypermethylation at the CpG island localized in the proximal promoter of the WRN gene is associated with a loss of RNA and protein levels and enzymatic activity, that can be reverted by the use of DNA demethylating agents.

Furthermore, the capacity of WRN to act as tumor suppressor gene is also underscored by the finding that the re-introduction of the gene in WRN-methylation deficient cells induces inhibition of cell growth in vitro and in vivo. But many exciting questions can now also be tackled in the laboratory and assessed in clinical samples, such as the links between WRN function and two critical components of the cellular machinery-disrupted in cancer cells: telomeres and p53. In the first case, the loss of WRN function has been proposed to facilitate the activation of the alternative lengthening of telomeres (ALT) mechanism, engendering cancer-relevant chromosomal aberrations and tumor formation in mouse models (Laud et al., 2005). Then, how do human cancer cells with WRN epigenetic inactivation behave with respect to their telomere length and telomerase activity? This question can also be applied to the classical tumor suppressor gene p53.

The induction of apoptosis by p53 is attenuated in WRN deficient cells (Spillare et al., 1999), and this phenotype cannot be rescued by other RecQ DNA helicases, such as BLM (Spillare et al., 2005). Thus, the attenuation of p53-mediated apoptotic pathway provides an additional explanation for the increased risk of cancer in WS patients. Furthermore, if in a given tumor we found a double hit in two caretakers of the genome, such as a point mutation in p53 and CpG island hypermethylation of WRN, these cancer cells may display a mutator phenotype characterized by a very high genomic instability. All these unanswered issues warrant further investigations.

Finally, it is worth emphasizing the potential clinical relevance of our findings. Our observations demonstrate that WRN hypermethylation renders these cancer cells very sensitive to the action of inhibitors of topoisomerase and DNA damaging agents. In human cancer cell lines of different types we have shown that these drugs induce a high apoptosis and chromosomal breakage rate when used against WRN hypermethylated cells. This is not just a pure observation restricted to laboratory models, but it can be translated to real patients. We have shown that the use of a chemotherapy agent with topoisomerase inhibition activity, irinotecan, is associated with increased overall survival in colorectal patients that display WRN promoter CpG island hypermethylation. Larger prospective studies are now necessary to expand these findings, but it is a line of translational research that merits further exploration. In this regard, there is a similar paradigmatic story in gliomas where the presence of epigenetic inactivation of another DNA repair gene, MGMT, is associated with increased responses to nitrosamides and their analogues (Esteller et al., 2000; Hegi et al., 2005). For the WRN gene, in addition to colon cancer, there is another tumor type, ovarian cancer, where another camptothecin analogue, topotecan, is one of the preferred used drugs and where WRN hypermethylation can “mark” those patients with enhanced clinical response to the chemotherapy.

In summary, our results suggest that the abolishment of the tumor suppressor functions of the progeroid WRN gene by epigenetic silencing is a relevant event in human tumorogenesis, associated with the generation of chromosomal instability; and, at the same time, it constitutes an Achilles' heel for these tumors, since they become more sensitive to the chemotherapeutic action of topoisomerase inhibitors and DNA damaging agents. Furthermore, these findings also represent another turn of the screw in the tight and long known connection between the aging and cancer processes.

### REFERENCES

All references mentioned throughout the specification are hereby incorporated in their entirety.
Agrelo R, Setien F, Espada J, Artiga M J, Rodriguez M, Perez-Rosado A, Sanchez-Aguilera A, Fraga M F, Piris M A, Esteller M. Inactivation of the lamin A/C gene by CpG island promoter hypermethylation in hematologic malignancies, and its association with poor survival in nodal diffuse large B-cell lymphoma. J Clin Oncol. 2005 Jun. 10; 23(17):3940-7.
Armes J E, Hammet F, de Silva M, Ciciulla J, Ramus S J, Soo W K, Mahoney A, Yarovaya N, Henderson M A, Gish K, Hutchins A M, Price G R, Venter D J. Candidate tumor-suppressor genes on chromosome arm 8p in early-onset and highgrade breast cancers. Oncogene. 2004 Jul. 22; 23(33):5697-702.
Brosh R M Jr, Karmakar P, Sommers J A, Yang Q, Wang X W, Spillare E A, Harris C C, Bohr V A. p53 Modulates the exonuclease activity of Werner syndrome protein. J Biol. Chem. 2001 Sep. 14; 276(37):35093-102.
Chen L, Oshima J. Werner Syndrome. J Biomed Biotechnol. 2002; 2(2):46-54.
Chen L, Lee L, Kudlow B A, Dos Santos H G, Sletvold O, Shafeghati Y, Botha E G, Garg A, Hanson N B, Martin G M, Mian I S, Kennedy B K, Oshima J. LMNA mutations in atypical Werner's syndrome. Lancet. 2003 Aug. 9; 362 (9382): 440-5.
Chughtai S A, Crundwell M C, Cruickshank N R, Affie E, Armstrong S, Knowles M A, Takle L A, Kuo M, Khan N, Phillips S M, Neoptolemos J P, Morton D G. Two novel regions of interstitial deletion on chromosome 8p in colorectal cancer. Oncogene. 1999 Jan. 21; 18(3):657-65.
Costello J F, Plass C. Methylation matters. J Med. Genet. 2001 May; 38(5):285-303.
Ershler W. B., Longo D L, Aging and cancer: issues of basic and clinical science. J. Natl. Cancer Inst. 89 (1997), pp. 1489-1497.
Epstein C J, Martin G M, Schultz A L, Motulsky A G. Werner's syndrome a review of its symptomatology, natural history, pathologic features, genetics and relationship to the natural aging process. Medicine (Baltimore). 1966 May; 45(3):177-221.
Esteller M, Silva J M, Dominguez G, Bonilla F, Matias-Guiu X, Lerma E, Bussaglia E, Prat J, Harkes I C, Repasky E A, Gabrielson E, Schutte M, Baylin S B, Herman J G. Promoter hypermethylation and BRCA1 inactivation in sporadic breast and ovarian tumors. J Natl Cancer Inst. 2000 Apr. 5; 92(7):564-9.
Esteller M, Garcia-Foncillas J, Andion E, Goodman S N, Hidalgo O F, Vanaclocha V, Baylin S B, Herman J G. Inactivation of the DNA-repair gene MGMT and the clinical response of gliomas to alkylating agents. N Engl J. Med. 2000 Nov. 9; 343(19):1350-4.
Esteller, M., Cordon-Cardo, C., Corn, P. G., Meltzer, S. J., Pohar, K. S., Watkins D. N., Capella, G., Peinado, M. A., Matias-Guiu, X., Prat, J., Baylin, S. B. and Herman, J. G. (2001). p14ARF silencing by promoter hypermethylation mediates abnormal intracellular localization of MDM2. Cancer Res., 61, 2816-2821.
Feinberg A P, Tycko B. The history of cancer epigenetics. Nat Rev Cancer. 2004 February; 4(2):143-53.
Goto M, Imamura O, Kuromitsu J. Matsumoto T, Yamabe Y, Tokutake Y, Suzuki N, Mason B, Drayna D, Sugawara M, Sugimoto M, Furuichi Y. Analysis of helicase gene mutations in Japanese Werner's syndrome patients. Hum Genet. 1997 February; 99(2):191-3.
Gray M D, Shen J C, Kamath-Loeb A S, Blank A, Sopher B L, Martin G M, Oshima J, Loeb L A. The Werner syndrome protein is a DNA helicase. Nat. Genet. 1997 September; 17(1):100-3.
Hegi M E, Diserens A C, Gorlia T, Hamou M F, de Tribolet N, Weller M, Kros J M, Hainfellner J A, Mason W, Mariani L, Bromberg J E, Hau P, Mirimanoff R O, Cairncross J G, Janzer R C, Stupp R. MGMT gene silencing and benefit from temozolomide in glioblastoma. N Engl J. Med. 2005 Mar. 10; 352(10):997-1003.
Herman J G, Umar A, Polyak K, Graff J R, Ahuja N, Issa J P, Markowitz S, Willson J K, Hamilton S R, Kinzler K W, Kane M F, Kolodner R D, Vogelstein B, Kunkel T A, Baylin S B. Incidence and functional consequences of hMLH1 promoter hypermethylation in colorectal carcinoma. Proc Natl Acad Sci USA. 1998 Jun. 9; 95(12):6870-5.
Herman, J. G. and Baylin, S. B. (2003). Gene silencing in cancer in association with promoter hypermethylation. N. Engl. J. Med., 349, 2042-2054.
Herranz M, Martin-Caballero J, Fraga M F, Ruiz-Cabello J. Flores J M, Desco M, Marquez V, Esteller M. The novel DNA methylation inhibitor zebularine is effective against the development of murine T-cell lymphoma. Blood 107, 1175-1177, 2006.
Hickson I D. RecQ helicases: caretakers of the genome. Nat Rev Cancer. 2003 March; 3(3):169-78.
Huang S, Li B, Gray M D, Oshima J, Mian I S, Campisi J. The premature ageing syndrome protein, WRN, is a 3′-->5′ exonuclease. Nat. Genet. 1998 October; 20(2):114-6.
Jones, P. A. and Laird, P. W. (1999). Cancer epigenetics comes of age. Nat. Genet., 1999 21, 163-167.
Laud P R, Multani A S, Bailey S M, Wu L, Ma J, Kingsley C, Lebel M, Pathak S, DePinho R A, Chang S. Elevated telomere-telomere recombination in WRN deficient, telomere dysfunctional cells promotes escape from senescence and engagement of the ALT pathway. Genes Dev. 2005 Nov. 1; 19(21):2560-70.
Lebel M, Leder P. A deletion within the murine Werner syndrome helicase induces sensitivity to inhibitors of topoisomerase and loss of cellular proliferative capacity. Proc Natl Acad Sci USA. 1998 Oct. 27; 95(22):13097-102.
Lebel M. Werner syndrome: genetic and molecular basis of a premature aging disorder. Cell Mol Life Sci. 2001 June; 58(7):857-67.
Lowe J, Sheerin A, Jennert-Burston K, Burton D, Ostler E L, Bird J, Green M H, Faragher R G. Camptothecin sensitivity in Werner syndrome fibroblasts as assessed by the COMET technique. Ann N Y Acad. Sci. 2004 June; 1019:256-9.
Matsumoto T, Shimamoto A, Goto M, Furuichi Y. Impaired nuclear localization of defective DNA helicases in Werner's syndrome. Nat. Genet. 1997 August; 16(4):335-6.
Nagy R, Sweet K, Eng C. Highly penetrant hereditary cancer syndromes. Oncogene. 2004 Aug. 23; 23(38):6445-70.
Okada M, Goto M, Furuichi Y, Sugimoto M. Differential effects of cytotoxic drugs on mortal and immortalized B-lymphoblastoid cell lines from normal and Werner's syndrome patients. Biol Pharm Bull. 1998 March; 21(3):235-9.
Opresko P L, Cheng W H, von Kobbe C, Harrigan J A, Bohr V A. Werner syndrome and the function of the Werner protein; what they can teach us about the molecular aging process. Carcinogenesis. 2003 May; 24(5):791-802.
Pichierri P, Franchitto A, Mosesso P, Palitti F. Werner's syndrome cell lines are hypersensitive to camptothecin-induced chromosomal damage. Mutat Res. 2000 Nov. 30; 456(1-2):45-57.
Ropero S, Setien F, Espada J, Fraga M F, Herranz M, Asp J, Benassi M S, Franchi A, Patino A, Ward L S, Bovee J, Cigudosa J C, Wim W, Esteller M. Epigenetic loss of the familial tumor-suppressor gene exostosin-1 (EXT1) disrupts heparan sulfate synthesis in cancer cells. Hum Mol. Genet. 2004 Nov. 15; 13(22):2753-65.
Salk D. Werner's syndrome: a review of recent research with an analysis of connective tissue metabolism, growth control of cultured cells, and chromosomal aberrations. Hum Genet. 1982; 62(1):1-5.
Spillare E A, Robles A I, Wang X W, Shen J C, Yu C E, Schellenberg G D, Harris C C. p53-mediated apoptosis is attenuated in Werner syndrome cells. Genes Dev. 1999 Jun. 1; 13(11):1355-60.
Spillare E A, Wang X W, von Kobbe C, Bohr V A, Hickson I D, Harris C C. Redundancy of DNA helicases in p53-mediated apoptosis. Oncogene. 2005 Nov. 14.
Suzuki N, Shimamoto A, Imamura O, Kuromitsu J, Kitao S, Goto M, Furuichi Y. DNA helicase activity in Werner's syndrome gene product synthesized in a baculovirus system. Nucleic Acids Res. 1997 Aug. 1; 25(15):2973-8.
Thweatt R, Goldstein S. Werner syndrome and biological ageing: a molecular genetic hypothesis. Bioessays. 1993 June; 15(6):421-6.
Yu C E, Oshima J, Fu Y H, Wijsman E M, Hisama F, Alisch R, Matthews S, Nakura J, Miki T, Ouais S, Martin G M, Mulligan J, Schellenberg G D. Positional cloning of the Werner's syndrome gene. Science. 1996 Apr. 12; 272(5259):258-62.
Yu C E, Oshima J, Wijsman E M, Nakura J, Miki T, Piussan C, Matthews S, Fu Y H, Mulligan J, Martin G M, Schellenberg G D. Mutations in the consensus helicase domains of the Werner syndrome gene. Werner's Syndrome Collaborative Group. Am J Hum Genet. 1997 February; 60(2):330-41.

1. A method for predicting the likelihood of successful treatment of cancer with a topoisomerase inhibitor and/or a DNA damaging agent and/or a DNA methyltransferase inhibitor and/or a HDAC inhibitor comprising:
(a) determining the methylation status of a RecQ helicase family gene in a sample obtained from a subject, wherein if the RecQ helicase family gene is methylated the likelihood of successful treatment is higher than if the RecQ helicase family gene is unmethylated; and/or
(b) determining the expression levels of a RecQ helicase family gene in a sample obtained from a subject, wherein a reduced level of expression of the RecQ helicase family gene indicates the likelihood of successful treatment is higher than if the RecQ helicase family gene is expressed at a higher level.

2. A method for predicting the likelihood of resistance to treatment of cancer with a topoisomerase inhibitor and/or a DNA damaging agent and/or a DNA methyltransferase inhibitor and/or a HDAC inhibitor comprising:
(a) determining the methylation status of a RecQ helicase family gene in a sample obtained from a subject, wherein if the RecQ helicase family gene is unmethylated the likelihood of resistance to treatment is higher than if the RecQ helicase family gene is methylated; and/or
(b) determining the expression levels of a RecQ helicase family gene in a sample obtained from a subject, wherein a normal level of expression of the RecQ helicase family gene indicates the likelihood of resistance to treatment is higher than if the RecQ helicase family gene is expressed at a lower level.

3. A method of selecting a suitable treatment regimen for cancer comprising:
(a) determining the methylation status of a RecQ helicase family gene in a sample obtained from a subject, wherein if the RecQ helicase family gene is methylated a topoisomerase inhibitor and/or a DNA damaging agent and/or a DNA methyltransferase inhibitor and/or a HDAC inhibitor is selected for treatment; and/or
(b) determining the expression levels of a RecQ helicase family gene in a sample obtained from a subject, wherein a reduced level of expression of the RecQ helicase family gene indicates a topoisomerase inhibitor and/or a DNA damaging agent and/or a DNA methyltransferase inhibitor and/or a HDAC inhibitor should be selected for treatment.

4. A method of diagnosing cancer comprising:
(a) determining the methylation status of a RecQ helicase family gene in a sample obtained from a subject, wherein methylation of the RecQ helicase family gene is indicative of cancer; and/or
(b) determining the expression levels of a RecQ helicase family gene in a sample obtained from a subject, wherein a reduced level of expression of the RecQ helicase family gene is indicative of cancer.

5. The method according to claim 1, wherein the RecQ family helicase gene comprises the WRN gene.

6. The method according to claim 1, wherein the promoter region of the gene is analysed in order to determine the methylation status.

7. The method according to claim 1, wherein the methylation status of the CpG island comprising the sequence according to SEQ ID NO: 1 is determined.

8. The method preceding according to claim 1, wherein the methylation status of the WRN gene is determined by methylation specific PCR, preferably real-time methylation specific PCR.

9. The method of claim 8 wherein the real-time methylation specific PCR comprises use of a probe selected from the group consisting of TAQMAN probes, MOLECULAR BEACONS probes, AMPLIFLUOUR primers, LIGHT-CYCLER, FRET probes, SCORPION primers.

10. (canceled)

11. (canceled)

12. The method according to any one of claim 1 wherein bisulphite sequencing is utilised in order to determine the methylation status of the WRN gene.

13. (canceled)

14. The method preceding according to claim 1, wherein expression levels are determined at the protein level and/or at the RNA level.

15. (canceled)

16. The method of claim 14 wherein expression levels are determined by real time detection.

17. A method of treating cancer in a subject comprising administration of a topoisomerase inhibitor, a DNA damaging agent, a DNA methyltransferase inhibitor, or a HDAC inhibitor, wherein the subject has been selected for treatment on the basis of the method of claim 1.

18. The method according to claim 1, wherein the topoisomerase inhibitor comprises a topoisomerase I inhibitor.

19. The method of claim 18 wherein the topoisomerase I inhibitor comprises camptothecin, or a derivative thereof.

20. The method of claim 19 wherein the derivative is selected from the group consisting of irinotecan (CPT-11), topotecan, lurtotecan and exatecan.

21. The method according to claim 1, wherein the DNA damaging agent is selected from the group consisting of a DNA interstrand cross linker, UV light, gamma irradiation and tritiated thymidine, and combinations thereof.

22. The method of claim 21 wherein the DNA interstrand cross linker is selected from the group consisting of mitomycin C, cis-platinum, 3,6-diaziridinyl-2,5-bis(carboethoxyamino)-1,4-benzoquinone (diaziquone, AZQ), melphalan and chlorambucil, and combinations thereof.

23. The method according to claim 1, wherein the DNA methyltransferase inhibitor is selected from the group consisting of antisense molecules, RNAi molecules and siRNA molecules which reduce expression of DNA methyltransferase genes, DNMT1, 5-azacytidine, 5-aza-2′-deoxycytidine, 5-fluouro-2′-deoxycytidine, pseudoisocytidine, 5,6-dihydro-5-azacytidine, 1-β-D-arabinofuranosyl-5-azacytosine, zebulaine, 5-azacytidine, Decitabine, L-ethionine, S-adenosyl-homocysteine, sinefungin, (S)-6-methyl-6-deaminosine fungin, 6-deaminosinefungin, N4-adenosyl-N4-methyl-2,4-diaminobutanoic acid, 5′-methylthio-5′-deoxyadenosine, 5′-amino-5′-deoxyadenosine and combinations thereof.

24. (canceled)

25. (canceled)

26. (canceled)

27. (canceled)

28. The method according to claim 1, wherein the HDAC inhibitor is selected from the group consisting of trichostatin A (TSA), suberoyl hydroxamic acid (SBHA), 6-(3-chlorophenylureido)caproic hydroxamic acid (3-Cl-UCHA), m-carboxycinnamic acid bishydroxylamide (CBHA), suberoylanilide hydroxamic acid (SAHA), azelaic bishydroxamic acid (ABHA), pyroxamide, scriptaid, aromatic sulfonamides bearing a hydroxamic acid group, oxamflatin, trapoxin, cyclic-hydroxamic-acid containing peptides, FR901228, MS-275, MGCD0103, short-chain fatty acids and N-acetyldinaline, and combinations thereof.

29. A method for treating cancer in a subject, said subject having a reduced level or activity of RecQ family helicase comprising reconstitution of RecQ family helicase activity in the subject.

30. The method of claim 29 wherein the RecQ family helicase comprises the WRN gene.

31. A method for treating cancer in a subject, said subject having a reduced level or activity of RecQ family helicase comprising reconstitution of RecQ family helicase activity in the subject, wherein the reduced level or activity of the RecQ family helicase is determined by the method of claim 1.

32. The method of claim 29 wherein reconstituting RecQ family helicase activity comprises delivery of wild type copies of the appropriate RecQ helicase family gene into the subject.

33. The method of claim 32 wherein delivery comprises use of a vector.

34. The method of claim 33 wherein the vector comprises an adenovirus.

35. The method according to claim 1, wherein the cancer is selected from the group consisting of from epithelial tumours, mesenchymal tumours and haematological malignancies.

36. The method of claim 35 wherein the epithelial tumour is selected from the group consisting of colorectal cancer, non-small cell lung cancer, gastric cancer, prostate cancer, breast cancer, ovarian cancer and thyroid cancer, preferably colorectal and ovarian cancer.

37. The method of claim 35 wherein the mesenchymal tumour is selected from the group consisting of chondrosarcomas and osteosarcomas.

38. The method of claim 35 wherein the haematological malignancy is selected from the group consisting of non-Hodgkin lymphoma, acute lymphoblastic leukaemia and acute myeloblastic leukaemia.

39. A kit for
(a) predicting the likelihood of successful treatment of cancer and/or the likelihood of resistance to treatment of cancer with a topoisomerase inhibitor and/or a DNA damaging agent and/or a DNA methyltransferase inhibitor and/or a HDAC inhibitor, and/or
(b) selecting a suitable treatment regimen for cancer and/or
(c) diagnosing cancer,
comprising carrier means containing therein a set of primers for use in detecting the methylation status of a RecQ family helicase gene.

40. The kit according to claim 39 wherein the RecQ family helicase comprises the WRN gene.

41. The kit according to claim 39 which further comprises a reagent which modifies unmethylated cytosine.

42. The kit according to claim 41, wherein the reagent comprises bisulfite.

43. The kit according to claim wherein the methylation status is determined by methylation specific PCR.

44. The kit according to claim 43 which further comprises probes for real-time detection of amplification products.

45. The kit according to claim 44 wherein the probes are also utilised to detect the methylation status of the RecQ family helicase gene.

46. The kit according to claim 39 wherein the kit further comprises a probe selected from the group consisting of TAQMAN probes, MOLECULAR BEACONS probes, AMPLIFLUOUR primers, LIGHT-CYCLER, FRET probes, SCORPION primers.

47. The kit according to claim 39, wherein the primers comprise primers for amplifying methylated DNA comprising the sequence set forth as SEQ ID NO: 1, as modified by bisulfite treatment.

48. The kit according to claim 39, wherein the primers comprise primers for amplifying unmethylated DNA comprising the sequence set forth as SEQ ID NO: 1, as modified by bisulfite treatment.

49. The kit according to claim 39, wherein the primers comprise primers comprising the nucleotide sequences set forth as SEQ ID NO: 4 and SEQ ID NO: 5 for the purposes of amplifying methylated DNA.

50. The kit according to claim 39 wherein the primers comprise primers comprising the nucleotide sequences set forth as SEQ ID NO: 2 and SEQ ID NO: 3 for the purposes of amplifying unmethylated DNA

51. The kit according to claim 39 wherein methylation status of the RecQ family helicase gene is determined using bisulphite sequencing.

52. The kit according to claim 51 wherein the bisulphite sequencing employs primers designed to direct sequencing across the promoter region of the RecQ family helicase gene.

53. The kit according to claim 51 wherein the bisulphite sequencing employs primers designed to direct sequencing across the DNA comprising the sequence set forth as SEQ ID NO: 1.

54. The kit according to claim 51 wherein the bisulphite sequencing employs primers that comprise the sequences set forth as SEQ ID NO: 6 and SEQ ID NO: 7.

55. The kit according to claim 39 which further comprises one or more control nucleic acid molecules of which the methylation status is known.

56. The kit of claim 55 further comprising both a methylated and unmethylated control nucleic acid molecule.

57. The kit of claim 56 wherein the unmethylated control nucleic acid molecule comprises a RecQ family helicase gene taken from non-cancerous cells.

58. The kit of claim 56 wherein the methylated control nucleic acid molecule comprises a RecQ family helicase gene taken from non-cancerous cells and which has been treated with a methyltransferase enzyme.

59. The kit according to claim 55 which further comprises primers for the amplification of control nucleic acid.

60. The kit of claim 59 wherein the primers for amplification of control nucleic acid are the same as those used to determine the methylation status of the RecQ family helicase gene.

61. The kit according to claim 57 wherein the RecQ family helicase gene comprises or consists of the WRN gene.

62. The kit according to claim 39, further comprising nucleic acid amplification buffer, enzymes to catalyze nucleic acid amplification, or a combination thereof.

63. (canceled)

64. Primers for amplifying methylated WRN DNA following treatment with bisulfite comprising the nucleotide sequences set forth as SEQ ID NO: 4 and SEQ ID NO: 5.

65. Primers for amplifying unmethylated WRN DNA following treatment with bisulfite comprising the nucleotide sequences set forth as SEQ ID NO: 2 and SEQ ID NO: 3.

66. Primers for bisulfite sequencing of the WRN gene comprising the sequences set forth as SEQ ID NO: 6 and SEQ ID NO: 7.

67. Primers which hybridise to the sequence set forth as SEQ ID NO: 1 following treatment with bisulfite.

68. (canceled)

69. (canceled)

70. The kit according to claim 58 wherein the RecQ family helicase gene comprises or consists of the WRN gene.

71. The kit according to claim 41, wherein the reagent comprises sodium bisulfite.
